(19) United States
(12) Patent Application Publication
(10) Pub. No.: US 2024/0117401 A1
(43) Pub. Date: Apr. 11, 2024

(54) METHODS OF ADJUSTING THE PH OF A CELL CULTURE MEDIUM

(71) Applicant: Bayer HealthCare LLC, Whippany, NJ (US)

(72) Inventors: R. Robert BALCARCEL, Fairfield, CA (US); Tam DUONG, San Ramon, CA (US)

(21) Appl. No.: 18/263,899

(22) PCT Filed: Feb. 11, 2022
(86) PCT No.: PCT/US2022/016113

§ 371 Date: Aug. 2, 2023

Related U.S. Application Data
(60) Provisional application No. 63/149,169, filed on Feb. 12, 2021.

(51) Int. Cl. C12P 21/02; C12N 5/00

(52) U.S. Cl. CPC C12P 21/02; C12N 5/00; C12N 2500/02; C12N 2500/32; C12N 2500/60

(57) ABSTRACT

Methods for adjusting the pH of a cell culture medium, methods for culturing cells in a pH-adjusted cell culture medium, and methods for making a polypeptide expressed by cells cultured in a pH-adjusted cell culture medium are described. Also described are systems for determining how much acid or base should be added to a cell culture medium to obtain a desired pH. The methods include a charge balance model that includes parameters for a functional relationship between a concentration of dissolved carbon dioxide in the cell culture medium, a mole fraction of gaseous carbon dioxide applied to the cell culture medium, a concentration of net medium acids in the cell culture medium, and the desired medium pH.

### CROSS-REFERENCE TO RELATED APPLICATION

This application claims priority to and the benefit of U.S. Provisional Application No. 63/149,169, filed Feb. 12, 2021, which is incorporated herein by reference for all purposes.

### FIELD OF THE INVENTION

Described herein are methods of adjusting the pH of a solution, such as a cell culture medium. Also described are methods of using the pH-adjusted cell culture medium, including methods of culturing cells and expressing a polypeptide from a cell cultured in the cell culture medium. Further described are systems for determining how the pH of a solution, such as a cell culture medium, should be adjusted.

### BACKGROUND

Over the past few decades, mammalian cell cultures have been employed to produce many valuable biopharmaceutical biologics. These are complex medicines made from living cells, including monoclonal antibodies, therapeutics proteins, and vaccines. Traditionally, biopharmaceutical drugs are released onto the market after having successfully met all the product quality standards needed for that therapeutic use. However, as an increasing number of complex biologic products cannot be fully characterized by “end product testing,” there is an added need for a far more thorough validation of the production process. The U.S. Food and Drug Administration (FDA) introduced Quality by Design (QbD) in this context where, based on an increased scientific knowledge base, quality standards are built into both the product and the process to meet specific objectives and mitigate potential risks during manufacturing. International Conference on Harmonisation, ICH Harmonised Tripartite Guideline: Pharmaceutical Development Q8(R2) (August 2009). QbD starts by identifying the critical process parameters (CPPs) and their effects on critical quality attributes (CQAs) and key performance indicators (KPIs). Given that the complex production processes include considerable variability, the experimentation that determines the relationship between CPPs with CQAs and KPIs is performed to define a range of conditions that effectively yield the required product CQAs. Rather than what had previously been a fixed process, this enhanced approach is intended to allow adjustments within the design space, including based on utilizing feedback from process analytical technologies so as to obtain increased control over the final product quality. This should reduce post-approval process changes and increase regulatory flexibility for biopharmaceutical companies, while ensuring therapeutic product safety, identity, purity and potency. Calcott, How QbD and the FDA Process Validation Guidance Affect Product Development and Operations, BioProcess International (2011).

Statistical design-of-experiments (DOEs) have been widely used as part of applying QbD concepts to recombinant protein production in mammalian cell culture processes. See Horvath et al., Characterization of a Monoclonal Antibody Cell Culture Production Process Using a Quality by Design Approach, Molecular Biotechnology, vol. 45, pp. 203-206 (2010); Kim et al., Applying the Quality by Design to Robust Optimization and Design Space Define for Erythropoietin Cell Culture Process, Bulletin of the Korean Chemical Society, vol. 40, no. 10, pp. 1002-1012 (2019); Nagashima et al., Application of a Quality by Design Approach to the Cell Culture Process of Monoclonal Antibody Production, Resulting in the Establishment of a Design Space, J. Pharmaceutical Sciences, vol. 102, no. 12, pp. 4274-4283 (2013); and Rouiller et al., Application of Quality by Design to the Characterization of the Cell Culture Process of an Fc-Fusion Protein, European J. Pharmaceutics and Biopharmaceutics, vol. 81, no. 2, pp. 426-437 (2012). These studies reported pH as one of the most important CPPs, with significant impacts on cell growth, productivity, and product quality. For example, for Chinese hamster ovary (CHO) cells, 2-fold higher growth rates were reported at pH 7.2 than at 6.85. Yoon et al., Effect on Culture pH on Erythropoietin Production by Chinese Hamster Ovary Cells Grown in Suspension at 32.5 and 37.0 degrees C., Biotechnology and Bioengineering, vol. 89, no. 3, pp. 345-356 (2005). Similarly, human PER.C6 cell growth was not affected in the range of pH 7.1-7.6 whereas there was a significant lag and lower growth rates at pH 6.8. Xie et al., Serum-Free Suspension Cultivation of PER.C6® Cells and Recombinant Adenovirus Production Under Different pH Conditions, Biotechnology and Bioengineering, vol. 80, no. 5, pp. 569-579 (2002). These influences of pH extend to the field of stem cell culture, with reported culture progenitor yields decreased more than 2-fold when the input medium pH was decreased from 7.3 to 7.0. Chaudhry et al., Culture pH and Osmolality Influence Proliferation and Embryoid Body Yields of Murine Embryonic Stem Cells, Biochemical Engineering J., vol., 45, no. 2, p. 126-135 (2009); Teo et al., Influence of Culture pH on Proliferation and Cardiac Differentiation of Murine Embryonic Stem Cells, Biochemical Engineering J., vol. 90, pp. 8-15 (2014). Thus, these studies highlight how for many cell types there are significant impacts from what can seem to be small differences in pH during experimentation to establish a well-understood design space. In particular, substantial effects of pH on recombination protein production, cell metabolism, and protein glycosylation are described extensively in the literature. See Borys et al., Culture pH Affects Expression Rates and Glycosylation of Recombinant Mouse Placental Lactogen Proteins by Chinese Hamster Ovary (CHO) Cells, Biotechnology, vol. 11, pp. 720-724 (1993); De Jesus et al., The Influence of pH on Cell Growth and Specific Productivity of Two CHO Cell Lines Producing Human Anti Rh D IgG, in: Lindner-Olsson E., Chatzissavidou N., Lüllau E. (eds) Animal Cell Technology: From Target to Market, ESACT Proceedings, vol. 1, Springer, Dordrech (2001); Kurano et al., Growth Behavior of Chinese Hamster Ovary Cells in a Compact Loop Bioreactor: 1. Effects of Physical and Chemical Environments, J. Biotechnology, vol. 15, pp. 101-11 (1990); Link et al., Bioprocess Development for the Production of a Recombinant MUC1 Fusion Protein Expressed by CHO-K1 Cells in Protein-Free Medium, J. Biotechnology, vol. 110, no. 1, pp. 51-62 (2004); Miller et al., A Kinetic Analysis of Hybridoma Growth and Metabolism in Batch and Continuous Suspension Culture: Effect of Nutrient Concentration, Dilution Rate, and pH, Biotechnology and Bioengineering, vol. 32, no. 8, pp. 947-965 (1988); Trummer et al., Process Parameter Shifting: Part I. Effect of DOT, pH, and Temperature on the Performance of Epo-Fc Expressing CHO Cells Cultivated in Controlled Batch Bioreactors, Biotechnology and Bioengineering, vol. 94, no. 6, pp. 1033-1044 (2006); Zanghi et al., Bicarbonate Concentration and Osmolality are Key Determinants in the Inhibition of CHO Cell Polysialylation Under Elevated pCO₂ or pH, Biotechnology and Bioengineering, vol. 65, no. 2, p. 182-191 (1999).

Addition of buffer is used to control the variation of pH during cell culture, and this is especially important for the high-throughput multi-well cultures that are commonly used initially in process development. For this purpose, the carbon dioxide (CO₂)/bicarbonate (HCO₃⁻) buffer system is routinely used. For fresh medium formulated with sodium bicarbonate, an equilibrium is reached between the HCO₃⁻ and dissolved CO₂ in the liquid phase, the latter also in equilibrium with the CO₂ level in the gas phase. Upon metabolism that produces lactic acid and CO₂ and other acidic and basic species, the pH of the culture is buffered by the transfer of CO₂ to or from the gas phase. Except in some cases where CO₂ accumulates to high levels at high cell concentrations (see Goudar et al., Decreased pCO₂ Accumulation by Eliminating Bicarbonate Addition to High Cell-Density Cultures, Biotechnology and Bioengineering, vol. 96, no. 6, pp. 1107-1117 (2006); Takuma et al., Dependence on Glucose Limitations of the pCO₂ Influences on CHO Cell Growth, Metabolism and IgG Production, Biotechnology and Bioengineering, vol. 97, no. 6, pp. 1479-1488 (2007)), the use of sodium bicarbonate does not negatively impact the physiology of the cells or their products, and so it is a widely used buffer for mammalian cell cultures.

Nonetheless, the use of sodium bicarbonate has its pitfalls since its buffering is dependent on the gas phase CO₂ concentrations, such that care needs to be taken when analyzing pH off-line since CO₂ degassing will increase the pH. This issue of CO₂ degassing due to low atmospheric CO₂ concentration also creates a challenge during cell culture medium preparation, since this normally takes place in an open environment under atmospheric conditions. The resulting continuously increasing pH during medium preparation is a problem compounded when many different culture media need to be prepared for DOE investigations, including many titrations to match the pHs of all the media. Also, this medium preparation usually takes place at room temperature while the cell culture process is close to 37° C. As pH is also temperature dependent, the amount of base/acid needed to achieve a target pH at room temperature will not yield an equivalent pH at the higher culture temperature.

The formulation of the cell culture medium and feeds is a critical step in cell culture process development. High-throughput technologies have enabled the acceleration of the screening of multiple formulations with multiple components simultaneously. Brühlmann et al., Parallel Experimental Design and Multivariate Analysis Provides Efficient Screening of Cell Culture Media Supplements to Improve Biosimilar Product Quality, Biotechnology and Bioengineering, vol. 114, no. 7, pp. 1448-1458 (2017); Lee et al., Development of a Serum-Free Medium for the Production of Erythropoietin by Suspension Culture of Recombinant Chinese Hamster ovary Cells Using a Statistical Design, J. Biotechnology, vol. 69, pp. 85-93 (1999); Jordan et al., Cell Culture Medium Improvements by Rigorous Shuffling of Components Using Media Blending, Cytotechnology, vol. 65, no. 1, pp. 31-40 (2013); Rouiller et al., A High-Throughput Media Design Approach for High Performance Mammalian Fed-Batch Cultures, MAbs, vol. 5, no. 3, pp. 501-511 (2013); Sandadi et al., Application of Fractional Factorial Designs to Screen Active Factors for Antibody Production by Chinese Hamster Ovary Cells, Biotechnology Progress, vol. 22, no. 2, p. 595-600 (2006). Though these experiments were well designed, they did not mention how the different formulations were prepared to minimize any variation in the pH of the parallel cultures. In addition to concerns regarding CO₂ degassing, the addition of some components could change the pH of these formulations. For example, most amino acids are not charged at physiological pH, but their unequal dissociation into cations and anions could still result in a slight change in the total charge of the species in solution thus leading to a slight difference in pH. This difference is greater for charged amino acids at physiological pH such as glutamic acid or aspartic acid. A titration process is often used to match the pHs of all the media, but this takes a lot more time when working with multiple solutions at once and does not address the challenges of working with bicarbonate-buffered media as described above for expressing polypeptides.

Therefore, there is a need for a semi-empirical model-based approach to medium formulation that does not depend on pH measurements at room temperature, to ease the assembly of multiple medium formulations and to provide increased accuracy and control of the medium pH under the culture environmental conditions.

Further, there is a need for a model to prescribe the amounts of base/acid addition needed to achieve a desired pH without the aid of the titration process to generate different cell culture medium with different combinations of amino acids for cells producing polypeptides.

Further, there is a need to provide a pH model and method to not only predict the pH of a salt buffered medium, but also to prescribe the exact amount of base/acid needed to achieve a desired pH without the aid of the titration process.

Further there is a need for a method or process for creating various different cell culture mediums with different combinations of amino acids that address the challenge with CO₂ evolution and temperature difference during the medium preparation and polypeptide expression processes.

### SUMMARY

Methods for adjusting the pH of a solution, such as a cell culture medium, methods for culturing cells in a pH-adjusted cell culture medium, and methods for making a polypeptide expressed by cells cultured in a pH-adjusted cell culture medium are described herein.

A method of adjusting the pH of a cell culture medium, can comprise: obtaining, for the cell culture medium, a functional relationship between a concentration of dissolved carbon dioxide in the cell culture medium and a mole fraction of gaseous carbon dioxide applied to the cell culture medium, and a concentration of net medium acids in the cell culture medium; adding carbonate salt or bicarbonate salt to the cell culture medium to obtain a desired carbonate salt or bicarbonate salt concentration in the cell culture medium; and determining, using a charge balance model, an amount of strong acid or strong base to be added to the cell culture medium to adjust the pH of the cell culture medium to a desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH.

The method may further comprise adding the determined amount of strong acid or strong base to the cell culture medium, thereby making a pH-adjusted cell culture medium.

In some implementations, the carbonate salt or the bicarbonate salt is sodium carbonate or sodium bicarbonate.

In some implementations, the method further comprises supplementing the cell culture medium with one or more ionic compounds, wherein the charge balance model is further based on the concentration of the one or more ionic compounds. In some embodiments, the one or more ionic compounds comprises one or more amino acids or ammonium chloride. In some embodiments, the one or more amino acids comprises glutamine, asparagine, or glutamic acid.

In some implementations of the described method, the strong base is sodium hydroxide. In some implementations of the described method, the strong acid is hydrochloric acid.

In some implementations of the described method, the charge balance model is defined by:

k*[M^{k+}]−[HCO₃⁻]−2*[CO₃²⁻]+[H⁺]−[OH⁻]−[NMA⁻]−[A⁻]+[B⁺]=0

wherein:
[M^{k+}] is a concentration of metal ions added as metal hydroxide, bicarbonate salt, or carbonate salt to the cell culture medium;
k is the charge of the metal ions;
[H⁺] is a concentration of protons in the cell culture medium needed to obtain the desired pH;
[OH⁻] is a concentration of hydroxide anions in the cell culture medium;
[NMA⁻] is the concentration of net medium acid ions in the cell culture medium;
[A⁻] is a concentration of negatively charged ions added to the cell culture medium, multiplied by the absolute value of their charge, excluding any OH⁻ or negatively charged ions included in [NMA−]; and
[B⁺] is a concentration of positively charged ions added to the cell culture medium, multiplied by the absolute value of their charge, excluding any H⁺, sodium ions included in [Na⁺], or positively charged ions included in [NMA−].

In some implementations of the described method, the charge balance model is defined by:

[Na⁺]−[HCO₃⁻]−2*[CO₃²⁻]+[H⁺]−[OH⁻]−[NMA⁻]−[A⁻]+[B⁺]=0

wherein:
[Na⁺] is a concentration of sodium ions added as sodium hydroxide, sodium bicarbonate, or sodium carbonate to the cell culture medium;
[H⁺] is a concentration of protons in the cell culture medium needed to obtain the desired pH;
[OH⁻] is a concentration of hydroxide anions in the cell culture medium;
[NMA⁻] is the concentration of net medium acid ions in the cell culture medium;
[A⁻] is a concentration of negatively charged ions added to the cell culture medium, multiplied by the absolute value of their charge, excluding any OH⁻ or negatively charged ions included in [NMA−]; and
[B⁺] is a concentration of positively charged ions added to the cell culture medium, multiplied by the absolute value of their charge, excluding any H⁺, sodium ions included in [Na⁺], or positively charged ions included in [NMA−].

In some implementations of the model,

$$[ {HCO}_{3}^{-} ] = \frac{{K}_{0} * {K}_{1} * P * {( {y CO}_{2} )}^{𝔪}}{100 * {K}_{H} * [ {H}^{+} ]}$$

$$[ {CO}_{3}^{2 -} ] = \frac{{K}_{0} * {K}_{1} * {K}_{2} * P * {( {y CO}_{2} )}^{𝔪}}{100 * {K}_{H} * {[ {H}^{+} ]}^{2}}$$

wherein:
K₀, K₁, and K₂ are dissociation constants for bicarbonate and carbonate anions;
P is a gas pressure applied to the cell culture medium;
yCO₂ is a molar percentage of CO₂ gas phase applied to the cell culture medium; and
m and K_{H} are each empirically determined parameters for the cell culture medium.

In some implementations of the described method, the concentration of net medium acids in the cell culture medium is modeled in the charge balance model as a function of pH of the cell culture medium. For example, the concentration of net medium acids in the cell culture medium may be modeled in a linear relationship with pH of the cell culture medium. In some embodiments, the concentration of net medium acids in the cell culture medium is modeled as:

[NMA⁻]=[C₀ₚ+C₁ₚ*(pH−7)]

wherein:
[NMA⁻] is the concentration of net medium acid ions in the cell culture medium; and
C₀ₚ and C₁ₚ are each empirically determined constants for the cell culture medium.

In some implementations of the described method, the concentration of net medium acids in the cell culture medium is modeled in the charge balance model as a function of temperature.

In some implementations of the described method, the concentration of net medium acids in the cell culture medium is modeled in the charge balance model as a function of pH and temperature.

In some implementations of the described method, the obtaining the functional relationship and the concentration of net medium acids for the charge balance model comprises empirically determining the functional relationship and the concentration of net medium acids of the cell culture medium. Empirically determining the functional relationship and the concentration of net medium acids of the cell culture medium may comprises, for example: measuring pH data for a plurality of conditions of the cell culture medium equilibrated at different gaseous carbon dioxide levels and containing different amounts of added strong acid or strong base; and fitting the charge balance model using the measured pH data. In some embodiments, the method comprises equilibrating the plurality of conditions at a desired culturing temperature prior to measuring the pH data. The desired culturing temperature may be, in some embodiments, about 35° C. to about 40° C.

In some implementations of the described method, the cell culture medium is prepared at room temperature.

In some implementations of the described method, the desired sodium carbonate or sodium bicarbonate concentration is about 1.5 g/L to about 2 g/L.

Also described herein is method of culturing cells, comprising adjusting the pH of a cell culture medium according to the above method; and culturing cells in the pH-adjusted cell culture medium. In some embodiments, the cells are mammalian cells, for example, Chinese hamster ovary (CHO) cells. In some embodiments, the cells are cultured in the cell culture medium at about 35° C. to about 40° C. In some embodiments, the cells are cultured in the cell culture medium under about 0.1% to about 20% mole fraction of CO₂. In some embodiments, the cells comprise a nucleic acid molecule encoding a recombinant polypeptide.

Also described herein is a method of producing a recombinant polypeptide, comprising culturing cells according to the above method, and producing the recombinant polypeptide in the pH-adjusted cell culture medium. In some embodiments, the recombinant polypeptide is an antibody or fragment thereof.

Also described herein is a system, comprising: one or more processors; and a memory communicatively coupled to the one or more processors and configured to store instructions that, when executed by the one or more processors, cause the system to: receive, at the one or more processors, for a cell culture medium, one or more parameters indicating a functional relationship between a concentration of dissolved carbon dioxide in the cell culture medium and a mole fraction of gaseous carbon dioxide applied to the cell culture medium, and a net medium acids parameter indicating a concentration of net medium acids in the cell culture medium; receive, at the one or more processors, a carbonate salt or bicarbonate salt parameter indicating a desired carbonate salt or bicarbonate salt concentration in the cell culture medium; receive, at the one or more processors, a pH parameter indicating a desired pH of the cell culture medium; and determine, using a charge balance model, an amount of strong acid or strong base to be added to the cell culture medium to adjust the pH of the cell culture medium to the desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH.

Further described herein is a method for producing a polypeptide in a host cell expressing the polypeptide, comprising culturing the host cell in a cell culture medium by preparing a cell culture medium with sodium bicarbonate to tightly control the pH of the medium, comprising: determining the excipients and relative amounts to be added to a cell culture medium to define a recipe; preparing a solution using the recipe and determining the pH of the solution to define a first data set; placing the solution in a CO₂ gassed and agitated bioreactor and allowing it to equilibrate to determine the resulting pH and pCO2 values to define a second data set; placing the solution in an incubator at a defined temperature and molar percent CO₂ and determining the pH and pCO₂ measurements to define a third data set; using the first, second, and third data sets and a pH model according to:

$$pH - p {K}_{a} - log [ B ] = log ( \frac{1}{s} ) - m * log ( {y}_{C {O}_{2}} )$$

to solve for the parameter values of m, s, and net medium acids simultaneously by minimizing:

Σ(k*[C^{k}])=0;

defining a target pH for the cell culture medium and adding an appropriate concentration of base to the cell culture medium as determined from the pH model to achieve the pH equivalence wherein the cell culture medium pH is tightly controlled; and producing the polypeptide.

In some embodiments, a salt is added to the solution to maintain the osmolality.

In some embodiments, the excipients are selected from the group consisting of glutamine, glutamate, asparagine, ammonium chloride, sodium chloride, and sodium hydroxide.

In some embodiments, the medium is placed in an incubator at 36.5° C. and 5% CO₂.

In some embodiments, the cell culture medium is prepared at room temperature.

In some embodiments, the pH of the cell culture medium is within 0.005 standard deviations of an expected pH value.

In some embodiments, the pH of the cell culture medium is 7.272+0.005.

In some embodiments, the method is automated.

In some embodiments, the method is performed in a batch fed process.

In some embodiments, the method is applicable at manufacturing scales and ensures robustness across scales.

In some embodiments, the method is applicable to ensure high quality comparison of multiple solutions with different amino acid additives during medium development or research.

In some embodiments, the method is applicable at small scale systems such as shake flasks.

Further described herein is a method for producing a polypeptide in a host cell expressing said polypeptide, comprising culturing the host cell in a production phase of the culture in a glutamine-free production culture medium, comprising: adding asparagine to the cell culture medium at a concentration in the range of 7.5 mM to 15 mM; adding aspartic acid to the cell culture medium at a concentration in the range of 1 mM to 10 mM; adding a salt to the cell culture medium; determining the excipients and relative amounts to be added to a cell culture medium to define a recipe; preparing a solution using the recipe and determining the pH of the solution to define a first data set; placing the solution in a CO₂ gassed and agitated bioreactor and allowing it to equilibrate to determine the resulting pH and pCO₂ values to define a second data set; placing the solution in an incubator at a defined temperature and molar percent CO₂ and determining the pH and pCO₂ measurements to define a third data set; using the first, second, and third data sets and a pH model according to:

$$pH - p {K}_{a} - log [ B ] = log ( \frac{1}{s} ) - m * log ( {y}_{C {O}_{2}} )$$

to solve for the parameter values of m, s, and net medium acids simultaneously by minimizing:

Σ(k*[C^{k}])=0;

defining a target pH for the cell culture medium and adding an appropriate concentration of base to the cell culture medium as determined from the pH model to achieve the pH equivalence wherein the cell culture medium pH is tightly controlled; and producing the polypeptide. In some embodiments, the method further comprises the step of isolating said polypeptide. In some embodiments, the production phase is a batch or fed batch culture phase. In some embodiments, the production medium is serum-free.

The embodiments provide methods for producing a polypeptide in a host cell expressing the polypeptide, comprising culturing the host cell in a cell culture medium by preparing a cell culture medium with sodium bicarbonate to tightly control the pH of the medium, comprising determining the excipients and relative amounts to be added to a cell culture medium to define a recipe, preparing a solution 1 using the recipe and determining the pH of the solution to define a first data set; placing solution 1 in a CO₂ gassed and agitated bioreactor and allowing it to equilibrate to determine the resulting pH and pCO2 values to define a second data set; placing solution 1 in an incubator at a defined temperature and % CO₂ and determining the pH and pCO₂ measurements to define a third data set; using the first, second, and third data sets and the pH model described herein to solve for the parameter values of m, s, and net medium acids simultaneously by minimizing a charge balance equation defining a target pH for the cell culture medium and adding an appropriate concentration of base to the cell culture medium as determined from the results to achieve the pH equivalence wherein the cell culture medium pH is tightly controlled; and producing the polypeptide.

### BRIEF DESCRIPTION OF THE DRAWINGS

The skilled artisan will understand that the drawings, described below, are for illustration purposes only. The drawings are not intended to limit the scope of the present teachings or claims in any way.

FIG. 1 shows and exemplary methods for adjusting the pH of a cell culture medium, according to some embodiments.

FIG. 2 shows an exemplary system that may be used to perform the methods described herein, according to some embodiments.

FIG. 3A shows CO₂ lost during solution preparation process affected pCO₂ of the solution as a function of time (pCO₂ vs. time). N=3, error bar=standard deviation.

FIG. 3B shows CO₂ lost during solution preparation process affected the pH of the solution as a function of time (pH vs. time). N=3, error bar=standard deviation.

FIG. 4 shows pH vs. Temperature. N=6, error bar=standard deviation.

FIG. 5 shows the amount of time it took to prepare one solution. N=5, error bar=standard deviation.

FIG. 6 shows pH of each solution after reaching equilibrium at 36.5° C. and 5% CO₂. N=5, error bar=standard deviation.

FIG. 7 shows equilibrium pH and pCO₂ data for 16 different solutions after a first experiment.

FIG. 8 shows the equilibrium pH and pCO₂ data for 16 different solution after a second experiment

FIG. 9 shows the relationship between pH and pCO2 for all 32 data points.

FIG. 10A shows a normality plot of empirical data collected according to Example 1.

FIG. 10B shows a residual plot of empirical data collected according to Example 1.

FIG. 11 shows measured pH data and model-fitted pH data, calculated from the charge balances using the determined parameters, according to an exemplary embodiment.

FIG. 12 shows measured pH data and model-fitted pH data, calculated from the charge balances using the determined parameters when considering net medium acids as a function of pH, according to an exemplary embodiment.

FIG. 13 shows measured pH and modeled pH for several culture medium with sodium hydroxide added to obtain a target pH, based on the charge balance model, according to an exemplary embodiment.

FIG. 14 shows measured pH and modeled pH for several culture medium with sodium hydroxide added to obtain a target pH, based on the charge balance model, according to another exemplary embodiment.

FIG. 15 shows measured pH versus model pH for redundant conditions of a cell culture medium measured using a first pH measurement device, according to some embodiments.

FIG. 16 shows measured pH versus model pH for redundant conditions of a cell culture medium measured using a second pH measurement device, according to some embodiments.

### DETAILED DESCRIPTION

This disclosure describes embodiments related to cell culture medium and methods of making the same. In particular, methods for adjusting the pH of a cell culture medium, methods for culturing cells in a pH-adjusted cell culture medium, and methods for making a polypeptide expressed by cells cultured in a pH-adjusted cell culture medium are described. Also described are systems for determining how much acid or base should be added to a cell culture medium to obtain a desired pH.

The methods and systems described herein are described in terms of a cell culture medium. One skilled in the art would recognize that the methods and systems described herein may be applied to any solution containing a carbonate or bicarbonate buffer.

The pH of a solution, such as a cell culture medium, can be adjusted by determining an amount of strong acid or strong base to be added to the cell culture medium to adjust the pH of the cell culture medium to a desired pH. This determination may be made using a charge balance model based on at least a functional relationship between the concentration of dissolved carbon dioxide in the cell culture medium and the mole fraction of gaseous carbon dioxide applied to the cell culture medium, a concentration of net medium acids in the cell culture medium, a desired carbonate salt or bicarbonate salt (such as sodium carbonate or sodium bicarbonate) concentration in the cell culture medium, and the desired pH. As further described herein, the functional relationship between the concentration of dissolved carbon dioxide in the cell culture medium and a mole fraction of gaseous carbon dioxide applied to the cell culture medium, and a concentration of net medium acids in the cell culture medium, may be empirically determined for a particular cell culture medium. These parameters (i.e., the functional relationship and the concentration of net medium acids) may be received by another entity (such as a manufacturer of the cell culture medium) or may be empirically determined by the end user. The method may further include adding the carbonate salt or the bicarbonate salt to the cell culture medium to obtain the desired carbonate salt or bicarbonate salt concentration in the cell culture medium.

Once the amount of strong acid or strong base to be added to the cell culture medium to adjust the pH of the cell culture medium to the desired pH is determined, the determined amount of strong acid or strong base may be added to the cell culture medium, thereby making a pH-adjusted cell culture medium.

The cell culture medium may be further supplemented with one or more ionic compounds (for example one or more amino acids and/or one or more salts). The charge balance model may be further based on the concentrations of the one more ionic compounds added to the cell culture medium.

The pH-adjusted cell culture medium may be used to culture cells. For example, a method of culturing cells may include adjusting the pH of the cell culture medium according the method described herein, and culturing cells in the pH-adjusted cell culture medium. The cells may include a nucleic acid molecule encoding a recombinant polypeptide. The recombinant polypeptide may be expressed by the cells in the cell culture medium. For example, a method of producing a recombinant polypeptide may include adjusting the pH of the cell culture medium according the method described herein, culturing cells comprising a nucleic acid molecule encoding a recombinant polypeptide in the pH-adjusted cell culture medium, and producing the recombinant polypeptide in the pH-adjusted cell culture medium.

Also described is a system or electronic device that includes one or more processors and a memory communicatively coupled to the one or more processors and configured to store instructions that, when executed by the one or more processors, cause the system or electronic device to determine an amount of strong acid or strong base to be added to the cell culture medium to adjust the pH of the cell culture medium to a desired pH. For example, the instructions may cause the system or electronic device to receive, at the one or more processors, a pH parameter indicating a desired pH of the cell culture medium; and determine, using a charge balance model, an amount of strong acid or strong base to be added to the cell culture medium to adjust the pH of the cell culture medium to the desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH.

### Definitions

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

Whenever appropriate, terms used in the singular will also include the plural and vice versa. The use of “a” herein means “one or more” unless stated otherwise or where the use of “one or more” is clearly inappropriate.

The use of “or” means “and/or” unless stated otherwise.

The use of “comprise,” “comprises,” “comprising,” “include,” “includes,” and “including” are interchangeable and are not limiting. Further, terms “such as,” “for example,” and “e.g.” are not intended to be limiting. For example, the term “including” shall mean “including, but not limited to.”

As used herein, the term “about” refers to +/−10% of the unit value provided.

As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term “substantially” is, therefore, used herein to capture the potential lack of completeness inherent in many biological and chemical phenomena.

The term “antibody” is used in the broadest sense and encompasses, in particular, individual monoclonal antibodies (including agonist and antagonist antibodies) and antibody compositions with polyepitopic specificity. The term “antibody” encompasses, in particular, monoclonal antibodies (including full-length monoclonal antibodies), polyclonal antibodies, multispecific antibodies (e.g., bispecific antibodies) and antibody fragments.

The term “net medium acids” or “NMA” refers to the net acidic content of a cell culture medium prior adding carbonate salt or bicarbonate salt or other medium supplement modeled by the charge balance model described herein, and prior to adjusting the pH of the cell culture medium. NMA is defined as a positive quantity for acidic species and negative for basic species. Hence, the concentration of NMA ([NMA⁻]) is net positive for an overall acidic medium, and is net negative for an overall basic medium.

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties.

### Charge Balance Model for Determining Cell Culture Medium pH

The carbon dioxide/bicarbonate system is routinely used as a buffer in mammalian cell culture medium. However, due to the continuous degassing of carbon dioxide and its dependence on temperature, it is difficult to achieve the target pH during preparation at ambient temperature and without control of dissolved carbon dioxide, and even more so at cell culture operating conditions. These problems were amplified when preparing multiple (e.g. 20) customized preparations of an in-sourced proprietary medium during research and process development of mammalian cell culture systems. Thus, a mathematical model was created to specify the amount of acid or base to add during preparation so as to achieve the target pH of each medium at process conditions without having to do titrations. The relationship between gaseous carbon dioxide and the dissolved carbon dioxide in the proprietary medium containing unknown species was specified using a modified Henry's Law equation. Further, to allow medium preparation without doing titration, the acid/base properties of the proprietary medium were fitted by a parameter related to its “net medium acids” (or “NIVIA”) during specification of model parameters. Besides being used to prepare the media, the model was further used to assess the equivalence of the pHs of the customized medium formulations despite variations in pCO₂ occurred during incubation and sampling.

The pH model (also referred to herein as the “charge balance model”) allows the pH of the cell culture medium to be predicted based on acids or bases added to the cell culture medium, or alternatively can indicate how much acid or base should be added to the cell culture medium based on a desired pH. As further explained herein, a coefficient indicating CO₂ solubility (e.g., s or K_{H}) and an exponential term indicating relationship between gas phase CO₂ and dissolved CO₂ (e.g., m) for a particular cell culture medium are constant for a given temperature, regardless of the amount of acid or base added to the cell culture medium. The coefficient and exponential can be used describe the functional relationship between a concentration of dissolved carbon dioxide in the cell culture medium and a mole fraction of gaseous carbon dioxide applied to the cell culture medium. The net medium acids may be modeled as a constant for a cell culture medium, or may be modeled as a function of pH, temperature, or both. In some embodiments, the concentration of net medium acids is held constant across different pH or temperature ranges. In some embodiments, the concentration of net medium acids is modeled as a function of pH. In some embodiments, the concentration of net medium acids is modeled as a function of temperature. In some embodiments, the concentration of net medium acids is modeled as a function of temperature and pH. The functional relationship may be empirically determined for a cell culture medium, for example at the desired operating temperature, before the pH of the cell culture medium is adjusted. Further, one can create several batches of a cell culture medium at different pHs using the same model constants.

The model initially assumes a charge balance of the medium to be dictated by the electroneutrality of the solution (i.e., a net charge of zero). Thus, the following had to be fulfilled:

Σ(k*[C^{k}])=0

where [C] is the concentration and k is the charge of each ion. The model further assumes a mass balance for each species in the cell culture medium, which can be provided by:

$${[ X ]}^{0} = {\sum }_{i = 1}^{i} [ {X}^{i +} ] + {\sum }_{i = 1}^{j} [ {X}^{j -} ] + [ X ]$$

where [X]⁰ is the initial concentration of compound X, Σᵢ₌₁ⁱ[Xⁱ⁺] is the summation of the concentration of all the cation species; Σ_{j =1}^{j}[X^{j-}] is the summation of the concentration of all the anion species; [X] is the concentration of the non-dissociated form.

Since the net charge of the cell culture medium is zero, the cell culture medium with any added bicarbonate salt or bicarbonate sale (e.g., sodium carbonate or sodium bicarbonate) and acid/base can be modeled using the following exemplary charge balance model:

[Na⁺]−[HCO₃⁻]−2*[CO₃²⁻]+[H⁺]−[OH⁻]−[NMA⁻]−[A⁻]+[B⁺]=0

In the exemplary charge balance model, [Na⁺] is a concentration of sodium ions added as sodium hydroxide, sodium bicarbonate, or sodium carbonate to the cell culture medium; [H⁺] is a concentration of protons in the cell culture medium needed to obtain the desired pH; [OH⁻] is a concentration of hydroxide anions in the cell culture medium; [A⁻] is a concentration of negatively charged ions added to the cell culture medium, multiplied by the absolute value of their charge, excluding any OH⁻ or negatively charged ions included in [NMA−]; and [B⁺] is a concentration of positively charged ions added to the cell culture medium, multiplied by the absolute value of their charge, excluding any H⁺, sodium ions included in [Na⁺], or positively charged ions included in [NMA−]. Without knowing the specific components and amounts (or concentrations) in a cell culture medium, the concentration of net medium acids can only be determined empirically. The model described herein includes a consideration of these net medium acids. This is particularly useful when purchasing proprietary cell culture media that need to have the pH adjusted by the end user. Further, even if components of the basal medium are known, the described model is substantially easier to use because not all the components in the culture medium need to be modeled. The number of components in a cell culture medium may be quite numerous (e.g., about 10 to about 30 components for bacterial and fungal cultures, or about 30 to about 100 components for mammalian cell cultures).

Although sodium bicarbonate or sodium carbonate systems are most commonly used in cell culture media, the model is applicable to any solution containing a carbonate salt or bicarbonate salt. Thus, the model may be considered for any carbonate salt as:

k*[M^{k+}]−[HCO₃⁻]−2*[CO₃²⁻]+[H⁺]−[OH⁻]−[NMA⁻]−[A⁻]+[B⁺]=0

wherein [M^{k+}] is a concentration of metal ions added as metal hydroxide, bicarbonate salt, or carbonate salt to the cell culture medium; k is the charge of the metal ions; [H⁺] is a concentration of protons in the cell culture medium needed to obtain the desired pH; [OH⁻] is a concentration of hydroxide anions in the cell culture medium; [NMA⁻] is the concentration of net medium acid ions in the cell culture medium; [A⁻] is a concentration of negatively charged ions added to the cell culture medium, multiplied by the absolute value of their charge, excluding any OH⁻ or negatively charged ions included in [NMA−]; and [B⁺] is a concentration of positively charged ions added to the cell culture medium, multiplied by the absolute value of their charge, excluding any H⁺, sodium ions included in [Na⁺], or positively charged ions included in [NMA−].

The carbon dioxide/bicarbonate buffer system includes the following equilibrium reactions (see Millero, Thermodynamics of the Carbon Dioxide System in the Oceans, Geochmica et Cosmochimica Acta, vol. 59, no. 4, pp. 661-677 (1995)):

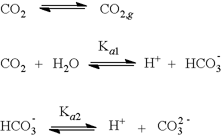

The concentration of anions and cations at equilibrium can be determined using the relationship provided by the dissociation constants. The first and the second dissociation constants of carbonic acid are given by:

$${K}_{a 1} = \frac{[ {H}^{+} ] [ {HCO}_{3}^{-} ]}{[ {CO}_{2} ]}$$

$${K}_{a 2} = \frac{[ {H}^{+} ] [ {CO}_{3}^{2 -} ]}{[ {HCO}_{3}^{-} ]}$$

Framed another way:

$$[ {HCO}_{3}^{-} ] = \frac{{K}_{0} * {K}_{1} * [ {CO}_{2} ]}{[ {H}^{+} ]}$$

$$[ {CO}_{3}^{2 -} ] = \frac{{K}_{0} * {K}_{1} * {K}_{2} * [ {CO}_{2} ]}{{[ {H}^{+} ]}^{2}}$$

Dissociation constants (e.g., Kₐ, K₀, K₁, K₂, etc.) for bicarbonate dissociation are known in the literature or can be empirically determined. For example, pKₐ values for the first and second dissociation constant at 36.5° C. were reported to be 6.303 and 10.238, respectively. Harned et al., The Ionization Constant of Carbonic Acid in Water and the Solubility of Carbon Dioxide in Water and Aqueous Salt solutions from 0 to 50°, J. American Chemical Society, vol. 65, no. 10, pp. 2030-2037 (1943). Thus, the values of Rₐ₁ and Kₐ₂ were determined to be 4.98×10⁻⁷ mol/L and 5.77×10⁻¹¹ mol/L, respectively.

At equilibrium and for a dilute system, the concentration of dissolved CO₂ is proportional to the mole fraction of CO₂ in the gas phase, as expressed by this version of the Henry's law:

[CO₂]=s*y_{co}_{₂}

where s (mM %) is a solubility factor that converts y_{co}_{₂} (percent partial pressure) into mIVI of CO₂ in the liquid phase. However, it has been shown that for cell culture media, this relationship is non-linear in such a way that cell culture media behave differently from a solution of water and bicarbonate. Thus, a modified form of Henry's law was developed:

[CO₂]=s*(y_{co}_{₂})^{m}

See Gramer et al., A Semi-Empirical Mathematical Model Useful for Describing the Relationship Between Carbon Dioxide, pH, Lactate and Base in a Bicarbonate-Buffered Cell Culture Process, Biotechnology and Applied Biochemistry, vol. 47, no. 4, pp. 197-204 (2007). The value “s” is therefore an exemplary coefficient indicating CO₂ solubility, and the value “m” is an exemplary exponential indicating CO₂ solubility. Practically, coefficient and exponential indicating CO₂ can be expressed in alternative expressions to suit the specific model. For example, when expressed in terms of total pressure (P) and Henry's law constant (k_{H});

$$[ {CO}_{2} ] = \frac{P * {( {y}_{{CO}_{2}} )}^{m}}{100 * {K}_{H}}$$

Thus, [HCO³⁻] and [CO₃²⁻] can be expressed as follows:

$$[ {HCO}_{3}^{-} ] = \frac{{K}_{0} * {K}_{1} * P * {( {y CO}_{2} )}^{m}}{100 * {K}_{H} * [ {H}^{+} ]}$$

$$[ {CO}_{3}^{2 -} ] = \frac{{K}_{0} * {K}_{1} * {K}_{2} * P * {( {y CO}_{2} )}^{m}}{100 * {K}_{H} * {[ {H}^{+} ]}^{2}}$$

The model may further account for the dissociation of water:

$$[ {OH}^{-} ] = \frac{{K}_{w}}{[ {H}^{+} ]}$$

wherein K_{w} is the dissociation constant of water.

Accordingly, the sum charge balance model for a cell culture medium may be written as:

$$[ {Na}^{+} ] - \frac{{K}_{0} {K}_{1} {P ( {y CO}_{2} )}^{m}}{100 {K}_{H} [ {H}^{+} ]} - \frac{2 {K}_{0} {K}_{1} {K}_{2} {P ( {y CO}_{2} )}^{m}}{100 {{K}_{H} [ {H}^{+} ]}^{2}} + [ \text{} {H}^{+} ] - \frac{{K}_{w}}{[ {H}^{+} ]} - [ {NMA}^{-} ] - [ {A}^{-} ] + [ {B}^{+} ] = 0$$

[Na⁺] is a concentration of sodium ions added as sodium hydroxide, sodium bicarbonate, or sodium carbonate to the cell culture medium; [H⁺] is a concentration of protons in the cell culture medium needed to obtain the desired pH; [OH⁻] is a concentration of hydroxide anions in the cell culture medium; [A⁻] is a concentration of negatively charged ions added to the cell culture medium, multiplied by the absolute value of their charge, excluding any OH⁻ or negatively charged ions included in [NMA−]; [B⁺] is a concentration of positively charged ions added to the cell culture medium, multiplied by the absolute value of their charge, excluding any H⁺, sodium ions included in [Na⁺], or positively charged ions included in [NMA−]; K₀, K₁, and K₂ are dissociation constants for bicarbonate and carbonate anions; P is a gas pressure applied to the cell culture medium; yCO₂ is a molar percentage of CO₂ gas phase applied to the cell culture medium; and m and K_{H} are each empirically determined parameters for the cell culture medium.

As discussed above, the values for K_{H}, m, and [NMA⁻] are constant at a specific temperature for a cell culture medium (although [NMA⁻] may optionally be modeled as a function of pH in a more refined model), regardless of the amount of sodium bicarbonate (or sodium carbonate) or acid or base added to the cell culture medium, and can be empirically determined. Further, the dissociation constants for water and carbonate/bicarbonate are known. Thus, this model function may be used to predict the pH of a cell culture medium based on the amount of bicarbonate salt or carbonate salt and strong acid or strong base added to the cell culture medium. Alternatively, and the more common usage, the amount of strong acid or strong acid that should be added to a cell culture medium, given a predetermined amount of sodium bicarbonate or sodium carbonate to be added and a desired pH, may be determined using the model.

The net medium acids in the cell medium may include weak acids and/or weak bases, and the equilibrium of these components may themselves be affected by the pH and/or temperature of the cell culture medium. Accordingly, in some embodiments, the concentration of net medium acids is modeled in the charge balance model as a function of pH of the cell culture medium. The relationship between the concentration of the net medium acids and pH may be a polynomial relationship, for example a linear relationship. In some embodiments, the concentration of net medium acids in the cell culture medium is modeled as:

[NMA⁻]=[C₀ₚ+C₁ₚ*(pH−7)]

wherein [NMA⁻] is the concentration of net medium acid ions in the cell culture medium; and C₀ₚ and C₁ₚ are each empirically determined constants for the cell culture medium. Thus, fully expanded, the charge balance model, in some embodiments, may be written as:

[Na⁺]−[HCO₃⁻]−2*[CO₃²⁻]+[H⁺]−[OH⁻]−[C₀ₚ+C₁ₚ*(pH−7)]−[A⁻]+[B⁺]=0

Or, in some embodiments:

$$[ {Na}^{+} ] - \frac{{K}_{0} {K}_{1} {P ( {y CO}_{2} )}^{m}}{100 {K}_{H} [ {H}^{+} ]} - \frac{2 {K}_{0} {K}_{1} {K}_{2} {P ( {y CO}_{2} )}^{m}}{100 {{K}_{H} [ {H}^{+} ]}^{2}} + [ \text{} {H}^{+} ] - \frac{{K}_{w}}{[ {H}^{+} ]} - [ {C}_{0 p} + {C}_{1 p} * ( pH - 7 ) ] - [ {A}^{-} ] + [ {B}^{+} ] = 0$$

In some instances, culture medium parameter (i.e., K_{H}, m, and [NMA⁻]) are affected by the specific method used to measure pH of the cell culture medium, for example due to variances among pH probes. Thus, parameter determination may done for each combination of basal medium with the particular methods used to measure pH during preparation.

### Empirical Determination of Model Parameters

The model parameters (i.e., K_{H}, m, and [NMA⁻]) may be determined in experiments conducted at the user-specified process temperatures. To empirically determine the parameters, the basal medium may be determined at different gaseous carbon dioxide levels and different amounts of added strong acid or strong base. At each of these conditions, the system from which the pH is measured is maintained at a equilibrium with gas phase CO₂ with the dissolved CO₂ in the liquid medium. For example, at a temperature of 36.5° C., the medium is equilibrated with yCO₂% is set at 4%, 6%, 8%, 10%, 15%, and 20% using mass flow controllers of a bioreactor system, for a medium preparations created with [HCl]=0 and [HCl]=5 mM. Parameters m, K_{H}, and [NMA⁻] are solved by a least-squares minimization method.

In some implementations, only one acid/base level is used along with two of more levels of yCO₂%, such that only m is can be determined first. Then, separately, where different levels of acid/base are tested, and given m from the first estimation, K_{H} and [NMA−] are then determined.

In another manner of considering the semi-empirical relationship between pH and pCO₂ for bicarbonate-buffered cell culture medium, the Henderson-Hasselbach equation may be used:

$$pH = {pK}_{a} + log ( \frac{[ B ]}{s * {( {y}_{{CO}_{2}} )}^{m}} ) ,$$

where pKa is 6.303 at 36.5° C. Sandadi et al., Application of Fractional Factorial Designs to Screen Active Factors for Antibody Production by Chinese Hamster Ovary Cells, Biotechnology Progress, vol. 22, no. 2, p. 595-600 (2006). For a standard carbonate pH equation, [B] is the HCO₃⁻ concentration (which maybe expressed, for example, in mM). For a proprietary medium with many unknown species, [B] can be defined as the summation of the concentrations of all acidic or basic species, besides bicarbonate and carbonate, added during medium preparation, along with net medium acids. These may include, for example, [Na⁺], [Gln⁺], [Asn⁺], [Glu⁺] as positive values, and [Net Medium Acid] as well as [Gln⁻], [Asn⁻], [Glu⁻], and [Glu⁽²⁻⁾] as negative values. As this term is insensitive to pH changes within the range of pH used in cell culture media, it is the same before and after equilibration with the gas phase CO₂.

To complete the description of the proprietary medium, a concentration of net medium acids can be considered as a counterbalance to the sodium hydroxide used in its preparation. Net medium acids may vary between different basal media that have different initial compositions. However, across cell media made using the same basal medium, net medium acid is constant.

The three empirical parameters describing a cell culture medium (e.g, m, s (or K_{H}), and net medium acids) may be found using the charge balance model equations for an un-supplemented preparation of medium measured at three conditions (1) during preparation at room temperature, (2) during equilibration at various gas phase CO₂ partial pressures at the target temperature, and (3) during equilibration at the target gas phase CO₂ concentration and temperature. For example, the Henderson-Hasselbach equation may be re-expressed as:

$$pH - p {K}_{a} - log [ B ] = log ( \frac{1}{s} ) - m * log ( {y}_{C {O}_{2}} )$$

This expression provides an exemplary relationship between y_{co}_{₂} and the pH of cell culture medium, using constants “m” and “s” as semi-empirical parameters. A plot of log (y_{co}_{₂}) vs. (pH−pKₐ−log[B]) provided a linear relationship with slope (−m) and an intercept of log(1/s).

After providing values for the concentration of all species and the three parameters, software (such as Microsoft™ Excel solver) may be used to find the pH value in this charge balance equation that minimizes it to zero.

In another example, the functional relationship between a concentration of dissolved carbon dioxide in the cell culture medium and a mole fraction of gaseous carbon dioxide applied to the cell culture medium (for example, parameters for K_{H} (or s) and m), and the concentration of net medium acids, for the charge balance model comprises using pH data measured from a plurality of conditions of the cell culture medium equilibrated at different gaseous carbon dioxide levels and different amounts of added strong acid or strong base. In some embodiments, the functional relationship and the concentration of net medium acids is determined simultaneously. In some embodiments, the functional relationship and the concentration of net medium acids is determined sequentially. The pH of the equilibrated conditions can be measured and used to parameterize the charge balance model. The charge balance for the plurality of conditions can be minimized, thereby providing the parameters for the functional relationship and [NMA⁻].

### Additional Supplements to Cell Culture Medium

The cull culture medium may be further supplemented with one or more additional additives, such as one or more salts, acids, or bases. For example, the cell culture medium may be supplemented with one or more amino acids and/or ammonium chloride. In some embodiments, the cell culture medium is supplemented with one or more amino acids. In some embodiments, the cell culture medium is supplemented with glutamine, asparagine, and/or glutamic acid. In some embodiments, the cell culture medium is supplemented with ammonium chloride. Advantageously, the concentration of net medium acids and the functional relationship between the concentration of dissolved carbon dioxide in the cell culture medium and the mole fraction of gaseous carbon dioxide applied to the cell culture medium (e.g., parameters m, K_{H}, and [NMA⁻] of the model) may be based on the cell culture medium prior to adding the additional charged species, and therefore generally do not need to be adjusted when adjusting the concentration of additives (unless the additive alter the functional relationship between dissolved and gaseous CO₂).

Charged species (for example, weak acids or bases) added to the cell culture medium can be accounted for using the charge balance model by incorporating the concentrations of the charged species, [A⁻] and [B⁺] in the charge balance model. For example, if the cell culture medium is supplemented with glutamine, asparagine, glutamic acid, and ammonium chloride, the charge balance model may be written as:

[Na⁺]−[HCO₃⁻]−2*[CO₃²⁻]+[H⁺]−[OH⁻]−[NMA⁻]+[Gln⁺]−[Gln⁻]+[Asn⁺]−[Asn⁻]+[Glu⁺]−[Glu⁻]−2*[Glu²⁻]+[NH₄⁺]−[CL⁻]=0

The amine (—NH₂), carboxyl (—COOH), and functional groups of an amino acid in aqueous medium are mostly protonated when the pH of the solution is below their respective pKa values. The concentrations of all the anions and cations at equilibrium can be determined using the pKₐ values. For example, in the case of glutamic acid, which has 3 pKₐ values, the following dissociation/association equilibria can be used:

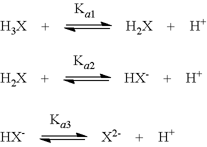

The dissociation/association equilibria between zwitterion and cation (Kₐ₁), zwitterion and anion (Kₐ₂), and anion and double-charged anion (Kₐ₃) can be described as:

$${K}_{a 1} = \frac{[ {H}_{2} X ] [ {H}^{+} ]}{[ {H}_{3} {X}^{+} ]}$$

$${K}_{a 2} = \frac{[ {H X}^{-} ] [ {H}^{+} ]}{[ {H}_{2} X ]}$$

$${K}_{a 3} = \frac{[ {X}^{2 -} ] [ {H}^{+} ]}{[ {H X}^{-} ]}$$

For monoacidic amino acids such as glutamine and asparagine, a third Kₐ value need not be considered.

Ammonium chloride is a soluble salt that releases an ammonium ion into solution, that when converted to ammonia releases another hydrogen ion.

NH₄Cl [symbol] NH₄⁺+Cl⁻

NH₄⁺+H₂O [symbol] NH₃+H₃O⁺

The dissociation equilibrium for ammonia was described as:

$${K}_{b} = \frac{[ {NH}_{3} ] [ {H}^{+} ]}{[ {NH}_{4}^{+} ]}$$

Values for dissociation constants of glutamine, asparagine, glutamic acid, and ammonia

at 36.5° C. are listed in Table 1.

TABLE 1
Dissociation constants at 36.5° C.

| Compound | Kₐ₁ (mol/L) | Kₐ₂ (mol/L) | Kₐ₃ (mol/L) | K_{b} (mol/L) |
|---|---|---|---|---|
| Glutamine | 7.05E−03 | 1.15E−09 | | |
| Asparagine | 7.31E−03 | 3.15E−09 | | |
| Glutamic acid | 6.58E−03 | 5.78E−05 | 4.16E−10 | |
| Ammonia | | | | 1.28E−09 |

See Kochergina et al., Influence of Temperature on the Heats of Acid-Base Reactions in L-Glutamine Aqueous Solution, Russian J. of Inorganic Chemistry, vol. 58, pp. 744-748 (2013); Kochergina et al., Thermochemical Study of Acid-Base Interactions in L-Asparagine Aqueous Solutions, Russian J. Inorganic Chemistry, vol. 56, no. 1481 (2011); Nagai et al., Temperature Dependence of the Dissociation Constant of Several Amino Acids, J. Chemical & Engineering Data, vol. 53, no. 3, pp. 619-627 (2008); Bates et al., Dissociation Constant of Aqueous Ammonia at 0 to 50° from E. m. f Studies of the Ammonium Salt of a Weak Acid, J. American Chemical Society, vol. 70, no. 3 pp. 1393-1395 (1950)

Accordingly, the cell culture medium supplemented with one or more ionic compound can be modeled using the charge balance model. In some embodiments, the one or more ionic compounds comprises ammonium chloride. In some embodiments, the one or more ionic compounds comprises an amino acid, such as an L-amino acid. In some embodiments, the one or more ionic compounds comprises one or more of glutamine, asparagine, and glutamic acid. Other supplemental components that may be added to the cell culture medium are known in the art and can be modeled according to the methods described herein. Other supplemental components may include, antifoaming agents, a poloxamer, salts, growth factors, serum, etc.

### Methods for Adjusting the pH of a Cell Culture Medium

The pH of a cell culture medium can be adjusted by determining an amount of strong acid or strong base to be added to the cell culture medium to adjust the pH of the cell culture medium to a desired pH. As discussed herein, this determination may be made using a charge balance model based on at least a functional relationship between the concentration of dissolved carbon dioxide in the cell culture medium and the mole fraction of gaseous carbon dioxide applied to the cell culture medium, a concentration of net medium acids in the cell culture medium, a desired carbonate salt or bicarbonate salt (such as sodium carbonate or sodium bicarbonate) concentration in the cell culture medium, and the desired pH. The method may further include adding the carbonate salt or the bicarbonate salt to the cell culture medium to obtain the desired carbonate salt or bicarbonate salt concentration in the cell culture medium. The method may further include adding the determined amount of strong acid or strong base to the cell culture medium, thereby making a pH-adjusted cell culture medium.

The empirical model parameters (e.g., the functional relationship between a concentration of dissolved carbon dioxide in the cell culture medium and a mole fraction of gaseous carbon dioxide applied to the cell culture medium, and the concentration of net medium acids in the cell culture medium) may be obtained through an empirical determination. Alternatively, the empirical model parameters may be received from another entity.

Thus, in some implementations of the method, the method of adjusting the pH of a cell culture medium includes obtaining, for the cell culture medium, a functional relationship between a concentration of dissolved carbon dioxide in the cell culture medium and a mole fraction of gaseous carbon dioxide applied to the cell culture medium, and a concentration of net medium acids in the cell culture medium; adding carbonate salt or bicarbonate salt to the cell culture medium to obtain a desired carbonate salt or bicarbonate salt concentration in the cell culture medium; and determining, using a charge balance model, an amount of strong acid or strong base to be added to the cell culture medium to adjust the pH of the cell culture medium to a desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH. The method may further include adding the determined amount of strong acid or strong base to the cell culture medium, thereby making a pH-adjusted cell culture medium.

In some implementations, the method of adjusting the pH of a cell culture medium includes receiving, for the cell culture medium, one or more parameters indicating a functional relationship between a concentration of dissolved carbon dioxide in the cell culture medium and a mole fraction of gaseous carbon dioxide applied to the cell culture medium, and a parameter indicating concentration of net medium acids in the cell culture medium; adding carbonate salt or bicarbonate salt to the cell culture medium to obtain a desired carbonate salt or bicarbonate salt concentration in the cell culture medium; and determining, using a charge balance model, an amount of strong acid or strong base to be added to the cell culture medium to adjust the pH of the cell culture medium to a desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH. The method may further include adding the determined amount of strong acid or strong base to the cell culture medium, thereby making a pH-adjusted cell culture medium.

In some embodiments, the method of adjusting the pH of a cell culture medium includes empirically determining, for the cell culture medium, a functional relationship between a concentration of dissolved carbon dioxide in the cell culture medium and a mole fraction of gaseous carbon dioxide applied to the cell culture medium, and a concentration of net medium acids in the cell culture medium; adding carbonate salt or bicarbonate salt to the cell culture medium to obtain a desired carbonate salt or bicarbonate salt concentration in the cell culture medium; and determining, using a charge balance model, an amount of strong acid or strong base to be added to the cell culture medium to adjust the pH of the cell culture medium to a desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH. The method may further include adding the determined amount of strong acid or strong base to the cell culture medium, thereby making a pH-adjusted cell culture medium.

The carbonate salt or bicarbonate salt is generally sodium bicarbonate or sodium carbonate, although in some embodiments a different carbonate or bicarbonate salt may be used. For example, in some embodiments, the carbonate salt or bicarbonate salt is magnesium carbonate, calcium carbonate, calcium-magnesium carbonate, potassium carbonate, zinc carbonate, iron carbonate, or other suitable carbonate or bicarbonate salts. The desired carbonate salt or bicarbonate salt concentration in the cell culture medium may depend on the specifications of the cell culture medium and/or manufacturer recommendations. In some embodiments, the desired sodium carbonate or sodium bicarbonate concentration is about 1.5 g/L to about 2 g/L, such as about 1.8 g/L.

The method may further comprise supplementing the cell culture medium with one or more ionic compounds. As further described herein, the charge balance model may be further based on the concentration of the one or more ionic compounds used to supplement the cell culture medium. For example, the cell culture medium may be supplemented with one or more amino acids and/or ammonium chloride. In some embodiments, the cell culture medium is supplemented with one or more amino acids. In some embodiments, the cell culture medium is supplemented with glutamine, asparagine, and/or glutamic acid. In some embodiments, the cell culture medium is supplemented with ammonium chloride.

The strong acid or strong base used to adjust the pH of the cell culture medium may be any suitable strong acid or strong base. Exemplary strong acids include chloric acid, hydrobromic acid, hydrochloric acid, hydroiodic acid, nitric acid, perchloric acid, phosphoric acid, and sulfuric acid. In some embodiments, the strong acid is hydrochloric acid. Exemplary strong bases include sodium hydroxide, potassium hydroxide, lithium hydroxide, rubidium hydroxide, cesium hydroxide, calcium hydroxide, barium hydroxide, and strontium hydroxide. In some embodiments, the strong base is sodium hydroxide.

The functional relationship and the concentration of net medium acids for the charge balance model may include empirically determining the functional relationship between the concentration of dissolved carbon dioxide in the cell culture medium and the mole fraction of gaseous carbon dioxide applied to the cell culture medium, and the concentration of net medium acids in the cell culture medium. For example, a plurality of samples of the cell culture medium may be equilibrated at different gaseous carbon dioxide levels and contain different amounts of strong acid or strong base. The pH of the samples, once equilibrated, can be measured, and the pH data fit to the charge balance model to determine the functional relationship and the concentration of net medium acids. The plurality of samples may include, for example, a first set of samples containing a first amount of added acid or base and equilibrated at different gaseous carbon dioxide levels, and a second set of samples containing a second amount of added acid or base (different from the first amount) and equilibrated at different gaseous carbon dioxide levels. The plurality of samples can be equilibrated at the desired operating temperature (i.e., culturing temperature) prior to measuring the pH of the plurality of samples. Because the functional relationship and the net medium acids may be temperature-dependent parameters, it is preferred to determine them at the operating temperature. Nevertheless, the medium may be prepared at a different temperature (e.g., room temperature, or about 25° C.).

In some embodiments, the desired culturing temperature is about 35° C. to about 40° C., such as about 36° C. to about 37° C., or about 36.5° C. In some embodiments, the culturing temperature is optimized to enhance mammalian cell growth. In some embodiments, the desired culturing temperature is about 25° C. to about 35° C., such as about 27° C. to about 32° C., or about 27° C. to about 30° C. In some embodiments, the culturing temperature is optimized to enhance insect cell growth. In some embodiments, the culturing temperature is optimized to enhance bacterial cell growth. In some embodiments, the culturing temperature is optimized to enhance virus replication.

In some embodiments, the cell culture medium is a serum-free medium.

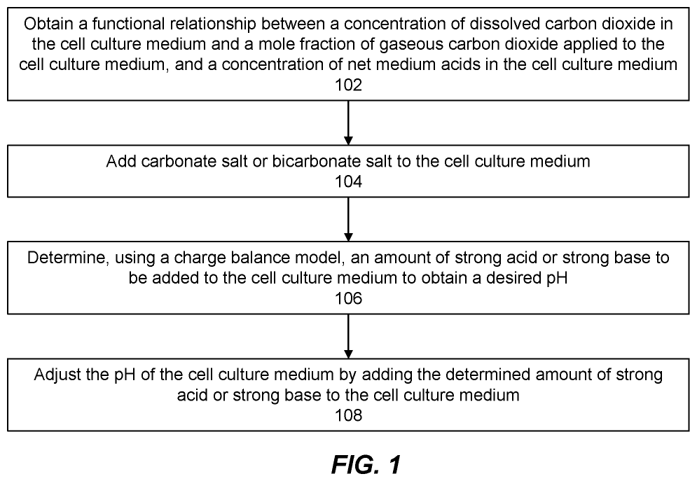

FIG. 1 shows and exemplary method of adjusting the pH of a cell culture medium. At 102, a functional relationship between the concentration of dissolved carbon dioxide in the cell culture medium and a mole fraction of gaseous carbon dioxide applied to the cell culture medium, and a concentration of net medium acids in the cell culture medium, are obtained. These parameters may be obtained, for example, by empirically determining the parameters or receiving the parameters from another entity. At 104, a carbonate salt or a bicarbonate salt (e.g., sodium carbonate or sodium bicarbonate) is added to the cell culture medium to obtain a desired carbonate or bicarbonate concentration in the cell culture medium. At 106, a charge balance model is used to determine an amount of strong acid or strong base to be added to the cell culture medium to obtain a desired pH for the cell culture medium. The charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH. At 108, the pH of the cell culture medium is adjusted by adding the determined amount of strong acid or strong base to the cell culture medium.

### Methods for Culturing Cells and Producing a Polypeptide

A method of culturing cells can include adjusting the pH of the cell culture medium according to the method described herein, and culturing cells in the pH-adjusted cell culture medium. For example, a method of culturing cells may include obtaining, for the cell culture medium, a functional relationship between a concentration of dissolved carbon dioxide in the cell culture medium and a mole fraction of gaseous carbon dioxide applied to the cell culture medium, and a concentration of net medium acids in the cell culture medium; adding carbonate salt or bicarbonate salt to the cell culture medium to obtain a desired carbonate salt or bicarbonate salt concentration in the cell culture medium; determining, using a charge balance model, an amount of strong acid or strong base to be added to the cell culture medium to adjust the pH of the cell culture medium to a desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH; adding the determined amount of strong acid or strong base to the cell culture medium, thereby making a pH-adjusted cell culture medium; and culturing cells in the pH-adjusted cell culture medium.

The cells cultured in the cell culture medium may be any suitable cell type. In some embodiments, the cells are mammalian cells, such as human cells. Exemplary mammalian cells may include FMK 293, 3T6, A49, A9, AtT-20, BALB/3T3, BHK-21, BHL-100, BT, Caco-2, Chang, CHO (e.g., CHO-K1), COS-1, COS-3, COS-7, CRFK, CV-1, D-17, Dauidi, GH1, GH3, H9, HaK, HCT-15, HeLa, HEp-2, HL-60, HT-1080, HT-29, HUVEC, I-10, IM-9, JEG-2, Jensen, Jurkat, K-562, KG-1, L2, LLC-WRC 256, McCoy, MCF7, WI-38, WISH, XC, and Y-1 cells. In some embodiments, the cells are CHO cells. In some embodiments, the cells are insect sells, such as Sf9, Sf21, or Schneider 2 (S2) cells. In some embodiments, the cells are bacterial cells, for example Escheichia coli cells. In some embodiments, the cells are plant cells. In some embodiments, the cells are yeast cells, such as Saccharomyces cerevisiae cells. In some embodiments, the cells are stem cells, such as human stem cells, or differentiated cell types.

The cells may be cultured at any suitable temperature, and may be selected, for example, based on the type of cell being cultured. In some embodiments, the culturing temperature is about 35° C. to about 40° C., such as about 36° C. to about 37 ° C., or about 36.5° C. In some embodiments, the culturing temperature is optimized to enhance mammalian cell growth. In some embodiments, the culturing temperature is about 25° C. to about 35° C., such as about 27° C. to about 32° C., or about 27° C. to about 30° C. In some embodiments, the culturing temperature is optimized to enhance insect cell growth. In some embodiments, the culturing temperature is optimized to enhance bacterial cell growth. In some embodiments, the culturing temperature is optimized to enhance virus replication.

One particular advantage of the methods described herein is that the empirical parameters for the charge balance model is that the parameters for the cell culture medium may be used for different mole fractions of CO₂ applied to the cell culture. Thus, the selected mole fraction of CO₂ can be changed as desired without needing to re-determine the model parameters. Thus, for example, if different cell lines are cultured in the same cell medium composition but at different mole fractions of CO₂, the model may be applied for each culture. In some implementations of the method, the cells are cultured in the cell culture medium under about 0.1% to about 20% mole fraction of CO₂, for example about 0.1% to about 0.5% mole fraction of CO₂, about 0.5% to about 1% mole fraction of CO₂, about 1% to about 2% mole fraction of CO₂, about 2% to about 5% mole fraction of CO₂, about 5% to about 10% mole fraction of CO₂, about 10% to about 15% mole fraction of CO₂, or about 15% to about 20% mole fraction of CO₂.

The cells cultured in the pH-adjusted cell culture medium may include a nucleic acid molecule encoding a polypeptide. For example, the cells may be host cells that include an expression vector encoding the polypeptide.

The pH-adjusted cell culture media described herein may be used in a method of culturing cells to produce polypeptides, such as antibodies or antibody fragments. The polypeptides produced by the cell cultured in the pH-adjusted cell culture medium may be homologous to the host cell, or preferably, may be exogenous, meaning that they are heterologous, i.e., foreign, to the host cell being utilized, such as a human protein produced by a Chinese hamster ovary cell, or a yeast polypeptide produced by a mammalian cell. In one variation, the polypeptide is a mammalian polypeptide directly secreted into the medium by the host cell. In another variation, the polypeptide is released into the medium by lysis of a cell comprising a nucleic acid encoding the polypeptide.

Any polypeptide that is expressible in a host cell may be produced in accordance with the present disclosure and may be present in the compositions provided. The polypeptide may be expressed from a gene that is endogenous to the host cell, or from a gene that is introduced into the host cell through genetic engineering. The polypeptide may be one that occurs in nature, or may alternatively have a sequence that was engineered or selected. An engineered polypeptide may be assembled from other polypeptide segments that individually occur in nature, or may include one or more segments that are not naturally occurring.

Polypeptides that may desirably be expressed in accordance with the present invention may be selected on the basis of an interesting biological or chemical activity. For example, the present invention may be employed to express any pharmaceutically or commercially relevant enzyme, receptor, antibody, hormone, regulatory factor, antigen, binding agent, etc.

Methods for producing polypeptides, such as antibodies, in cell culture are well known in the art. Provided herein are non-limiting exemplary methods for producing an antibody (e.g., full length antibodies, antibody fragments and multispecific antibodies) in cell culture. The methods herein can be adapted by one of skill in the art for the production of other proteins, such as protein-based inhibitors.

Generally, the cells are combined (contacted) with any of the cell culture media under one or more conditions that promote any of cell growth, maintenance and/or polypeptide production. Methods of culturing a cell and producing a polypeptide employ a culturing vessel (bioreactor) to contain the cell and cell culture medium. The culturing vessel can be composed of any material that is suitable for culturing cells, including glass, plastic or metal. Typically, the culturing vessel will be at least 1 liter and may be 10, 100, 250, 500, 1000, 2500, 5000, 8000, 10,000 liters or more. Nevertheless, other sized containers may be used, for example test tubes, microchips, multi-well plates, or flasks of other sizes, such as 250 mL, 100 mL, 50 mL, 25 mL, 15 mL, 10 mL, or smaller. The culture conditions, such as temperature, pH, and the like, are those previously used with the host cell selected for expression, and will be apparent to the ordinarily skilled artisan. Culturing conditions that may be adjusted during the culturing process include but are not limited to pH and temperature.

A cell culture is generally maintained in the initial growth phase under conditions conducive to the survival, growth and viability (maintenance) of the cell culture. The precise conditions will vary depending on the cell type, the organism from which the cell was derived, and the nature and character of the expressed polypeptide.

The temperature of the cell culture in the initial growth phase will be selected based primarily on the range of temperatures at which the cell culture remains viable. For example, during the initial growth phase, CHO cells grow well at 37° C. In general, most mammalian cells grow well within a range of about 25° C. to 42° C. Preferably, mammalian cells grow well within the range of about 35° C. to 40° C. Those of ordinary skill in the art will be able to select appropriate temperature or temperatures in which to grow cells, depending on the needs of the cells and the production requirements.

The cell culture may be agitated or shaken during the initial culture phase in order to increase oxygenation and dispersion of nutrients to the cells. In accordance with the present invention, one of ordinary skill in the art will understand that it can be beneficial to control or regulate certain internal conditions of the bioreactor during the initial growth phase, including but not limited to temperature, oxygenation, etc.

An initial culturing step is a growth phase, wherein batch cell culture conditions are modified to enhance growth of recombinant cells, to produce a seed train. The growth phase generally refers to the period of exponential growth where cells are generally rapidly dividing, e.g. growing. During this phase, cells are cultured for a period of time, usually, but not limited to, 1 to 4 days, e.g. 1, 2, 3, or 4 days, and under such conditions that cell growth is optimal. The determination of the growth cycle for the host cell can be determined for the particular host cell by methods known to those skilled in the art.

In the growth phase, a basal culture medium provided herein and cells may be supplied to the culturing vessel in batch. The culture medium in one aspect contains less than about 5% or less than 1% or less than 0.1% serum and other animal-derived proteins. However, serum and animal-derived proteins can be used if desired. At a particular point in their growth, the cells may form an inoculum to inoculate a culture medium at the start of culturing in the production phase. Alternatively, the production phase may be continuous with the growth phase. The cell growth phase is generally followed by a polypeptide production phase.

During the polypeptide production phase, the cell culture may be maintained under a second set of culture conditions (as compared to the growth phase) conducive to the survival and viability of the cell culture and appropriate for expression of the desired polypeptide. For example, during the subsequent production phase, CHO cells express recombinant polypeptides and proteins well within a range of 25° C. to 38° C. Multiple discrete temperature shifts may be employed to increase cell density or viability or to increase expression of the recombinant polypeptide or protein. In one aspect, a medium as provided herein reduces the presence of metabolic by-products when used in a method of increasing polypeptide production as compared to contaminants obtained when the polypeptide is produced in a different medium. In one variation, the contaminants are reactive oxygen species. In one aspect, a medium as provided herein reduces color intensity of a polypeptide product when used in a method of increasing production of the polypeptide as compared to color intensity obtained when the polypeptide product is produced in a different media. In one variation, a method of increasing polypeptide production comprises a temperature shift step during the polypeptide production phase. In a further variation, a temperature shift step comprises a shift of the temperature from 31° C. to 38° C., from 32° C. to 38° C., from 33° C. to 38° C., from 34° C. to 38° C., from 35° C. to 38° C., from 36° C. to 38° C., from 31° C. to 32° C., from 31° C. to 33° C., from 31° C. to 34° C., from 31° C. to 35° C., or from 31° C. to 36° C.

The cells may be maintained in the subsequent production phase until a desired cell density or production titer is reached. In one embodiment, the cells are maintained in the subsequent production phase until the titer to the recombinant polypeptide reaches a maximum. In other embodiments, the culture may be harvested prior to this point. For example, the cells may be maintained for a period of time sufficient to achieve a viable cell density of 1, 5, 10, 15, 20, 25, 30, 35, 40, 45, 50, 55, 60, 65, 70, 75, 80, 85, 90, 95 or 99 percent of maximal viable cell density. In some cases, it may be desirable to allow the viable cell density to reach a maximum, and then allow the viable cell density to decline to some level before harvesting the culture.

The polypeptide of interest preferably may recovered from the culture medium as a secreted polypeptide, or may be recovered from host cell lysates when directly expressed without a secretory signal. In one aspect, the polypeptide produced is an antibody, such as a monoclonal antibody.

The culture medium or lysate may be centrifuged to remove particulate cell debris. The polypeptide thereafter may be purified from contaminant soluble proteins and polypeptides, with the following procedures being exemplary of suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG. A protease inhibitor such as phenyl methyl sulfonyl fluoride (PMSF) also may be useful to inhibit proteolytic degradation during purification. One skilled in the art will appreciate that purification methods suitable for the polypeptide of interest may require modification to account for changes in the character of the polypeptide upon expression in recombinant cell culture. Polypeptides can be generally purified using chromatographic techniques (e.g., protein A, affinity chromatography with a low pH elution step and ion exchange chromatography to remove process impurities). For antibodies, the suitability of protein A as an affinity ligand depends on the species and isotype of any immunoglobulin Fc domain that is present in the antibody.

Other methods for expressing and isolating polypeptides, including recombinant polypeptides, are known in the art.

### Computer Systems and Electronic Devices

The methods described herein may include the use of electronic device or system for implementing the methods. For example, the electronic device or system may be used to determine or fit one or more model parameters of the charge balance model for determining the amount of acid or base that should be added to the cell culture medium.

By way of example, a system or electronic device may include one or more processors; and a memory communicatively coupled to the one or more processors and configured to store instructions that, when executed by the one or more processors, cause the system to: receive, at the one or more processors, for a cell culture medium, one or more parameters indicating a functional relationship between a concentration of dissolved carbon dioxide in the cell culture medium and a mole fraction of gaseous carbon dioxide applied to the cell culture medium, and a net medium acids parameter indicating a concentration of net medium acids in the cell culture medium; receive, at the one or more processors, a carbonate salt or bicarbonate salt parameter indicating a desired carbonate salt or bicarbonate salt concentration in the cell culture medium; receive, at the one or more processors, a pH parameter indicating a desired pH of the cell culture medium; and determine, using a charge balance model, an amount of strong acid or strong base to be added to the cell culture medium to adjust the pH of the cell culture medium to the desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH.

A user may use such as system or electronic device, for example, to determine how much acid or base should be added to cell culture medium to obtain the desired pH at the desired operating temperature.

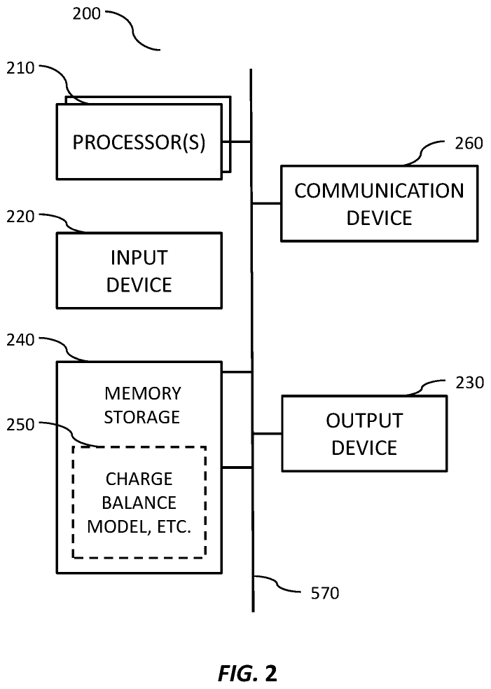

FIG. 2 illustrates an example of a computing device or system in accordance with one embodiment. Device 200 can be a host computer connected to a network. Device 200 can be a client computer or a server. As shown in FIG. 2, device 200 can be any suitable type of microprocessor-based device, such as a personal computer, workstation, server or handheld computing device (portable electronic device) such as a phone or tablet. The device can include, for example, one or more processor(s) 210, input devices 220, output devices 230, memory or storage devices 240, and communication devices 260. Software 250 residing in memory or storage device 240 may comprise, e.g., an operating system as well as software for executing the methods described herein. Input device 220 and output device 230 can generally correspond to those described herein, and can be either connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker. The input device 220 and the output device 230 can be the same device or different devices.

Storage 240 can be any suitable device that provides storage (e.g., an electrical, magnetic or optical memory including a RAM (volatile and non-volatile), cache, hard drive, or removable storage disk). Communication device 260 can include any suitable device capable of transmitting and receiving signals over a network, such as a network interface chip or device. The components of the computer can be connected in any suitable manner, such as via a wired media (e.g., a physical system bus 280, Ethernet connection, or any other wire transfer technology) or wirelessly (e.g., Bluetooth®, Wi-Fi®, or any other wireless technology).

Software module 250, which can be stored as executable instructions in storage 240 and executed by processor(s) 210, can include, for example, an operating system and/or the processes that embody the functionality of the methods of the present disclosure (e.g., as embodied in the devices as described herein).

Software module 250 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 240, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can be implemented using any operating system, e.g., an operating system suitable for operating on the network. Software module 250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 210.

### EXAMPLES

### Example 1

The basal medium used in this study was a chemically defined, proprietary medium from Cytiva (Massachusetts, USA). It was a custom order of ActiCHOP medium where glutamine, glutamic acid, and asparagine were removed. These three amino acids along with ammonia chloride were added to the custom medium individually or in combinations. This custom ActiCHOP medium also has 1.8 g/L of sodium bicarbonate per manufacture's recipe. To obtain all 16 solutions at a pH of 7.27 at 36.5° C. and an osmolality of 330 mOsm/kg, different amounts of 5N sodium hydroxide and 5M sodium chloride were added as determined by the pH model presented below. All 16 solutions were then placed in shake flasks within a humidified incubator, at 36.5° C., 5% CO₂ and 125 rpm. After equilibrium was reached (1-2 days), samples were taken and their pH and pCO₂ levels measured.

pH and pCO₂ measurements were performed using a Siemens RapidLab 248 blood gas analyzer, unless otherwise stated. pCO₂ is the equilibrium partial pressure of the gas phase CO₂ and was reported in terms of mm Hg. pCO₂ was converted to y_{co}_{₂} (%) using a factor of 100%/760 mm Hg.

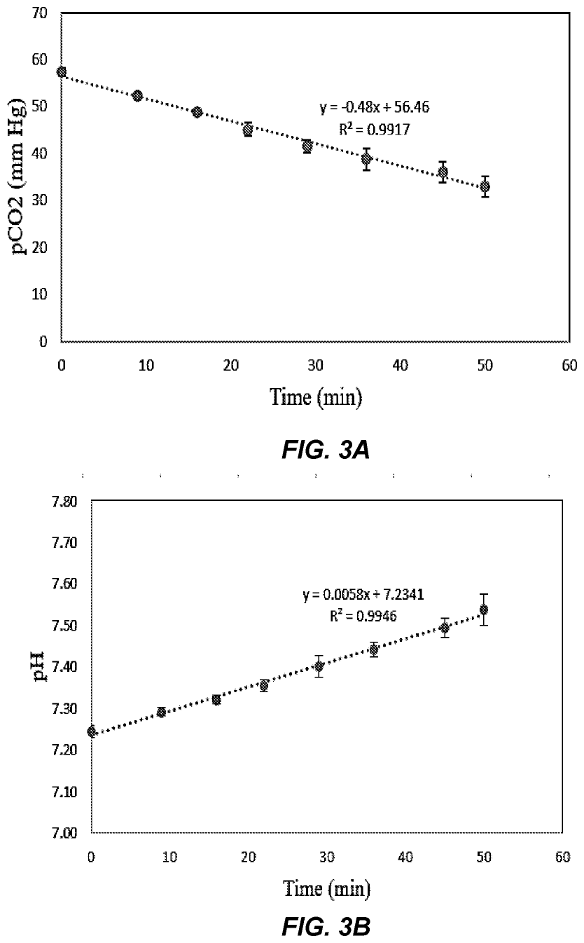

pCO₂ decreased over time indicating degassing of CO₂ occurred during medium preparation process In order to demonstrate CO₂ degassing during the medium preparation process, an experiment was carried out during the preparation of one of the medium solutions above. The solution was prepared in a 100 ml flask and was mixed at speed 5 setting on Cimarec Basic stir plate (Thermo Scientific, Massachusetts, USA). The flask was not capped, and samples were taken over a period of 50 minutes, which is a typical amount of time to prepare a single solution. The pH and pCO₂ of each sample were recorded. FIG. 3A shows that pCO₂ dropped linearly over time at a rate of −0.48 mmHg/min; at the same time, the pH of the solution increased linearly over time at a rate of 0.0058 pH unit per minute. See FIG. 3B. After 50 minutes, pCO₂ dropped by 24 mm Hg whereas pH increased by 0.29 unit. This result confirmed the challenge expected during solution preparation of medium containing bicarbonate buffer. As CO₂ was continuously gassing out, the pH of the solution was increasing and thus, the amount of base/acid needed for the titration process could depend on the exact timing and the extent of degassing from experiment to experiment.

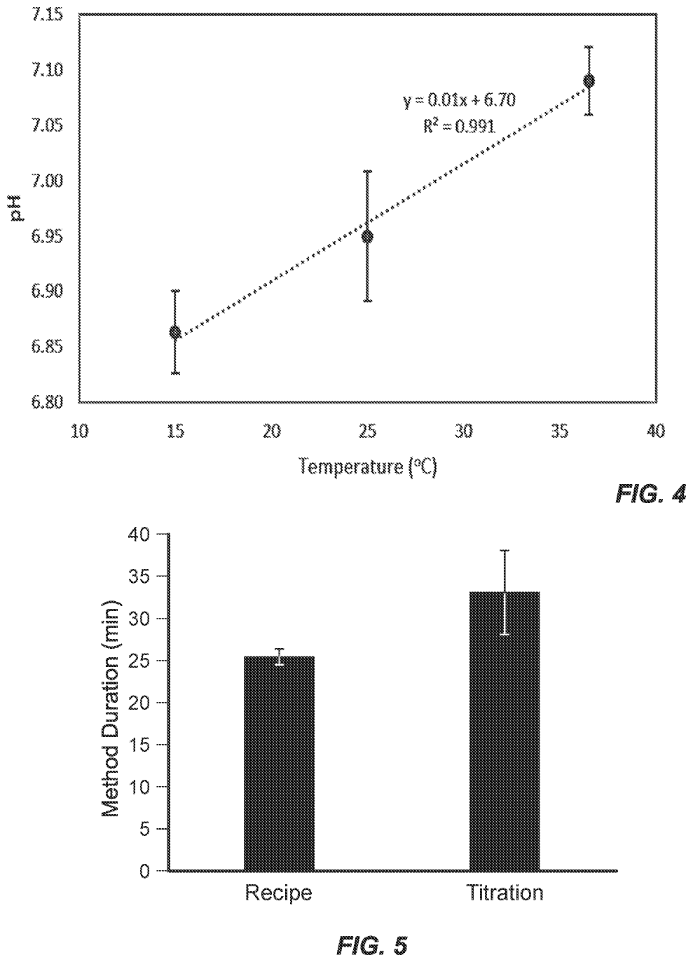

To demonstrate the relationship between pH and temperature of the medium solution, an experiment was carried out using AMBR250 bioreactors (Sartorius, Aubagne, France) filled with custom ActiCHOP medium. The gas flow rates were set to achieve concentrations of 10% CO₂ and 90% air, dry basis. Temperature setpoint was changed subsequently from 36.5° C. to 25° C., then 15° C. A sample was taken after each system reached steady state. The sample was analyzed for pH using Orion VersaStar Pro benchtop meter (Thermo Scientific). As shown in FIG. 4 below, for every 10 degree increase in temperature, pH increased by 0.1 unit when gas phase CO₂ was kept at a concentration 10% of the total gas flow rate.

This relationship between pH and temperature was not as expected. It had been shown before in the case of water with bicarbonate, that as temperature increased, pH decreased. Green, Effect of Temperature on pH of Alkaline Waters—Waters Containing Carbonate, Bicarbonate, and Hydroxide Alkalinity, vol. 41, no. 8, pp. 1795-797 (1949). This finding again supported the theory that cell culture medium could be different from a solution of just water and bicarbonate, possibly because cell culture medium could have contained other buffers and components that potentially affected its physical properties.

The m, s, and net medium acids for the 16 solutions were taken to be the same as these different solutions were established based on the same basal medium. First, a solution of ActiCHOP only (solution 1) was prepared from powder as instructed per manufacturer's protocol. The pH of this solution was recorded. Solution 1 was then placed into 4×2-litre bioreactors. The total gas flow rate was set at 200 ccm (air, nitrogen, and CO₂), agitation at 200 revolution/min and temperature at 36.5° C. The composition of CO₂ in the inlet was set at values in a range of approx. 4%-20% of total gas flow rate. After steady state was reach at each % CO₂ level, a sample was taken for pH and pCO₂ measurement. Lastly, solution 1 was placed into the incubator at 36.5° C. and 5% CO₂. The pH and pCO₂ measurements were recorded again after the solution reached equilibrium. Using these 3 sets of data and the pH model developed in section 2.4, values of m, s, and net medium acids were found simultaneously by minimizing Equation (20). Parameter m was found to be 0.827±0.021, s was 0.540±0.026 mM/%CO₂; and net medium acids was 0.033 M for this ActiCHOP solution.

The motivation to develop the pH model came from the anticipated longer time needed for and foreseeable challenges of preparing 16 different solutions at the same pH using titrations. An experiment involved preparing solution 1 was carried out, using both the titration method and recipe method that used the pH model to predict the exact amount of base needed. FIG. 5 below shows how long each method took to prepare this solution.

The average amount of time it took to prepare solution 1 using the recipe method was 8 minutes less than the average time needed using the titration method. The titration method also had more variation in the amount of time needed, which was due to the extra time needed to achieve the right pH with the addition of base/acid. Furthermore, the total time to prepare this solution 5 times for the recipe method was only 1 hour and a half, compared to the total time to 2 hours and a half using the titration method. Also, the recipe method allowed multiple solutions to be made in parallel by a single person whereas the titration method required more manual handling during the titration step.

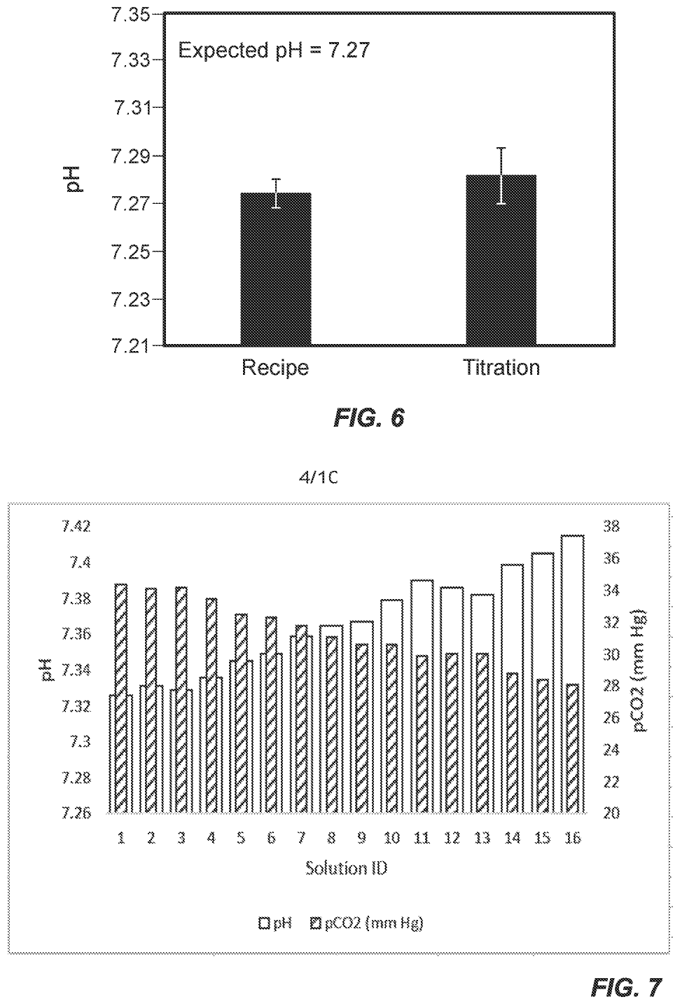

After the solutions were made using both methods, they were placed into two different bioreactors and set at 36.5° C. with a gas phase composition of 5% CO₂. pH data is shown in FIG. 6.

The expected pH was 7.27 at 36.5° C. and 5% CO₂. The pH of the solutions made by the recipe method was 7.274±0.005; whereas the pH of the solutions made by the titration method was 7.282±0.009. Truly, both methods achieved the desired target pH within 0.012 units, which is quite remarkable. It appears that the challenges of doing a titration at room temperature and with additional time to degas CO₂ were not evident here. This is perhaps the result of having prepared small volumes with excellent mixing, on the same day in succession, and with extreme care in weighing reagents and titration with close monitoring of pH. Despite this, the standard deviation for the titration method, albeit still relatively small in magnitude, was almost two times bigger than that of the recipe method. In contrast, experience from medium preparation using titration in manufacturing demonstrates a standard deviation that is 0.10 units, ten times higher. Thus, altogether, it would still be beneficial to carry out the medium solution preparation using the recipe method.

With m, s, and net medium acids determined using solution 1, the pH model with temperature and pH targets of 36.5° C. and 5% CO₂ at equilibrium was used to determine the exact amount of base added to achieve equivalent pH across all 16 media. The target pH was 7.27. Sodium chloride was also added to each solution respectively to ensure equivalent osmolality. Table 2 below details the amount of each component added to make up each solution.

TABLE 2
Amount of chemical added to each solution
Amount added per 100 mL medium

| | Solution: | | | | | | | | |
|---|---|---|---|---|---|---|---|---|---|
| | 1 | 2 | 3 | 4 | 5 | 6 | 7 | 8 | 9 |
| Glutamine (g) | 0 | 0.088 | 0 | 0 | 0.088 | 0.088 | 0 | 0.088 | 0 |
| Glutamate (g) | 0 | 0 | 0 | 0.044 | 0 | 0.044 | 0.044 | 0.044 | 0 |
| Asparagine (g) | 0 | 0 | 0.066 | 0 | 0.066 | 0 | 0.066 | 0.066 | 0 |
| NH₄Cl (g) | 0 | 0 | 0 | 0 | 0 | 0 | 0 | 0 | 0.011 |
| 5M NaCl (μL) | 210 | 150 | 160 | 150 | 100 | 90 | 100 | 40 | 170 |
| 5N NaOH (μL) | 0 | 2 | 4 | 62 | 6 | 64 | 65 | 66 | 0 |

| | Solution: | | | | | | |
|---|---|---|---|---|---|---|---|
| | 10 | 11 | 12 | 13 | 14 | 15 | 16 |
| Glutamine (g) | 0.088 | 0 | 0 | 0.088 | 0.088 | 0 | 0.088 |
| Glutamate (g) | 0 | 0 | 0.044 | 0 | 0.044 | 0.044 | 0.044 |
| Asparagine (g) | 0 | 0.066 | 0 | 0.066 | 0 | 0.066 | 0.066 |
| NH₄Cl (g) | 0.011 | 0.011 | 0.011 | 0.011 | 0.011 | 0.011 | 0.011 |
| 5M NaCl (μL) | 110 | 120 | 110 | 60 | 50 | 60 | 0 |
| 5N NaOH (μL) | 2.2 | 3.4 | 61 | 4.4 | 62 | 63 | 64 |

After being made, the solutions described were transferred to 250 ml Erlenmeyer flasks with a vent cap (Corning, New York, USA) and placed in an incubator at 36.5 degrees Celsius and 5% CO₂ (38 mm Hg pCO₂). Each flask was taken out of the incubator for pH and pCO₂ measurements, one at a time starting with flask 1. FIG. 7 represents the equilibrium pH and pCO₂ for all 16 solutions at the first experiment.

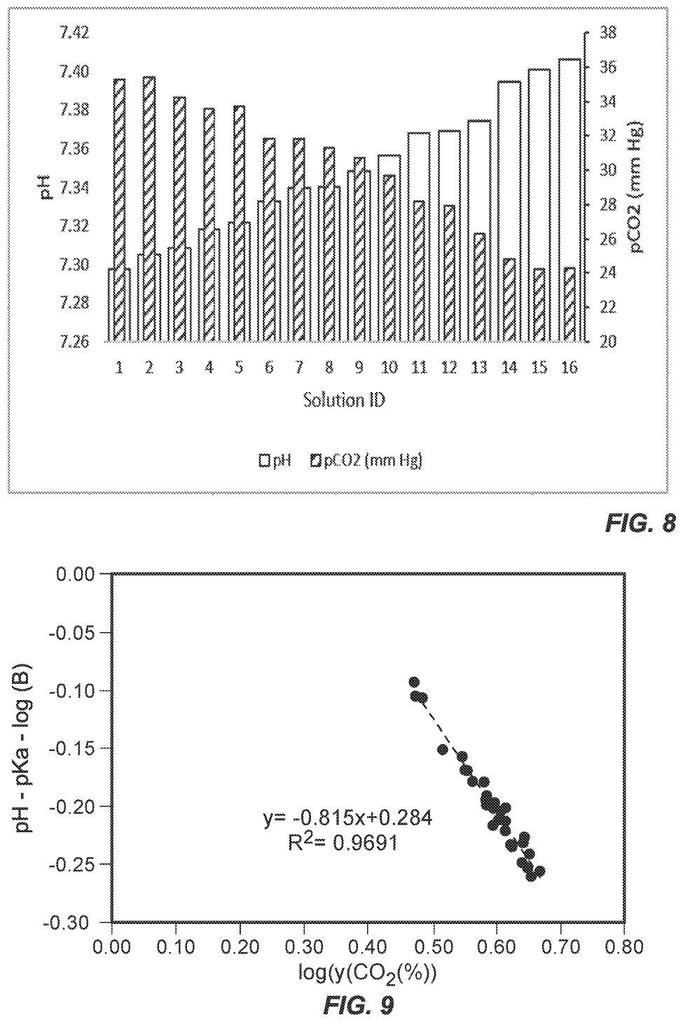

The pH of solution 1 was 7.33, 0.06 unit higher than expected where its pCO₂ was 34.4 mm Hg, 3.6 mm Hg lower than expected. This difference could be due to the fluctuation of the CO₂ level inside the incubator right before the sample was taken. The pHs of all subsequent solutions were higher whereas their pCO₂ were lower compared to the values of solution 1. This trend was observed again after the second experiment was repeated, as shown in FIG. 8.

A possible explanation for these observations could be that the CO₂ level inside the incubator was no longer the same every time the incubator door was open to sample a flask. These observations made it difficult to conclude that these 16 different solutions achieved the same pH using the recipe method. However, as mentioned in section 2.3, there was a relationship between pH and pCO₂ of these solutions. Thus, the pH and pCO₂ data of all 16 solutions over 2 experiments were fitted with Equation 18, shown in FIG. 9.

The slope (−m) was found to be −0.815±0.054, whereas the intercept of log(1/s) was found to be 0.284±0.032, with parameter s calculated to be 0.052±0.032 mM/% (the reported errors were based on the 95% confidence interval). These two values were not significantly different from the pH model parameters calculated in section 3.3 (p>0.05). The data is shown in Table 3 below.

TABLE 3
Comparison of parameters obtained
from equilibrium data vs. model.

| Parameter | Equilibrium data | Model |
|---|---|---|
| m | 0.815 ± 0.054 | 0.827 ± 0.021 |
| s | 0.520 ± 0.032 | 0.540 ± 0.026 |

The values of the parameters from the equilibrium data were in agreement with the parameters from the model. The model stipulated a true equilibrium whereas the experimental data might not have been truly at equilibrium given that disturbances were present (incubator CO₂ cycling, incubator door opening between sample, etc.). Nevertheless, the pH model developed provided 16 different solutions that were observed to have different pH and pCO₂ from raw data, but were in fact still related the model. This means that, at the exact same concentration of gas phase CO₂, the pH of each solution would be similar.

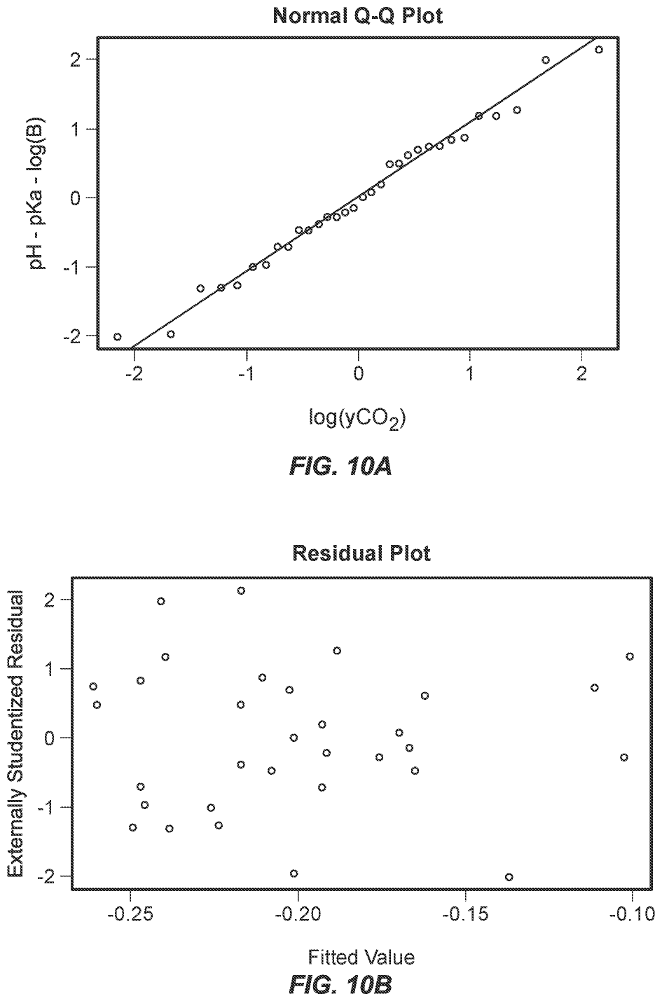

The R² value for the line for FIG. 9 was found to be 0.969 with an adjusted R² value of 0.968. The normal probability and the residual plot were graphed using R Studio program. The normal probability plot in FIG. 10A shows that all data was found to be on a straight line. The residual plot in FIG. 10B has no obvious pattern and all the externally studentized residual values were within the ±2 region. Thus, it can be concluded that this dataset had met the normality and constant variance assumptions and the fit for this model was a good fit.

Cell culture media plays an important role in cellular growth, metabolism, and productivity. pH is a CPP thus it is critical to control medium's pH tightly. Sodium bicarbonate is a popular buffer used in cell culture media, but the preparation of cell culture medium with sodium bicarbonate had many challenges; these challenges are addressed using the methods described herein. A pH model was used to provide a recipe for each of 16 different medium formulations, thus enabling the preparation of these media at room temperature without titration and to meet a pH target of 7.27 at equilibrium with 5% gas phase CO₂ at 36.5° C. However, due to the difficulty with pH/pCO₂ measurements of samples from flasks taken from an incubator, pH and pCO₂ of these 16 solutions were not the same. Nevertheless, their pH and pCO₂ were demonstrated to be related through a modified Henderson-Hasselbach equation. The pH model described herein also would help enable the automation of the solution preparation process, especially in the case of multiple solutions are needed. Though these methods are most useful at small scales, they are also applicable at manufacturing scales and could help ensure process robustness across scales.

### Example 2

This example demonstrates parameter determination for the charge balance model.

A proprietary medium (“Medium A”) was prepared according to the manufacturer's protocol. Medium A was provided as a powder, which was dissolved in water forming the basal liquid medium. To the basal liquid medium, 6.5 mL of NaOH was added, then 1.8 g/L of sodium bicarbonate followed by the final amount of water. Finally, the pH of the medium at room temperature (15-25° C.) was titrated to a pH within the range of 6.90 to 7.55 using a pH probe and additional amounts of NaOH or HCl.

Each of two 3 L Applikon Bioreactors were filled with two liters of the prepared medium (Bioreactor 1 and Bioreactor 2). 30 mL of 5 N hydrochloric acid was added to the medium in the second bioreactor (Bioreactor 2).

The temperature of the bioreactors was set to 36.5° C. and the medium agitated at 200 rpm. Gas flow containing air and CO₂ was supplied to the bioreactors. Eight compositions of inlet gas were used: carbon dioxide in the inlet gas streams were 1, 2, 4, 6, 8, 10, 15, and 20%. For each composition of gas, the bioreactors were operated to reach steady state prior to sampling for pH, which was measured using a Siemens RAPIDLab® blood gas analyzer.

The measured pH at each of 16 steady states is reported in Table 4.

TABLE 4

| | | | Measured | Modeled | |
|---|---|---|---|---|---|
| Sample | Bioreactor | yCO₂ % | pH | pH | Difference |
| 1 | 1 | 1 | 7.22 | 7.23 | 0.01 |
| 2 | 1 | 2 | 7.63 | 7.75 | 0.12 |
| 3 | 1 | 4 | 7.59 | 7.55 | −0.04 |
| 4 | 1 | 6 | 7.41 | 7.35 | −0.06 |
| 5 | 1 | 8 | 7.19 | 7.15 | −0.04 |
| 6 | 1 | 10 | 7.06 | 7.08 | 0.02 |
| 7 | 1 | 15 | 6.92 | 6.96 | 0.05 |
| 8 | 1 | 20 | 6.87 | 6.88 | 0.07 |
| 9 | 2 | 1 | 6.63 | 6.23 | −0.13 |
| 10 | 2 | 2 | 6.93 | 7.10 | 0.17 |
| 11 | 2 | 4 | 6.85 | 6.90 | 0.05 |
| 12 | 2 | 6 | 6.73 | 6.70 | −0.04 |
| 13 | 2 | 8 | 6.65 | 6.58 | −0.07 |
| 14 | 2 | 10 | 6.58 | 6.49 | −0.09 |
| 15 | 2 | 15 | 6.52 | 6.43 | −0.09 |
| 16 | 2 | 20 | 6.41 | 6.31 | −0.10 |

The charge balance model was implemented for each of 16 samples in Table 1. The

general form of this equations was as follows

$$[ {Na}^{+} ] - \frac{{K}_{0} * {K}_{1} * P * {( {y CO}_{2} )}^{m}}{100 * {K}_{H} * [ {H}^{+} ]} - \frac{2 * {K}_{0} * {K}_{1} * {K}_{2} * P * {( {y CO}_{2} )}^{m}}{100 * {K}_{H} * {[ {H}^{+} ]}^{2}} + [ {H}^{+} ] - [ {OH}^{-} ] - [ {NMA}^{-} ] - [ {Cl}^{-} ] = 0$$

Since no additional supplements were added to the cell culture medium, the [A⁻] and [B⁺] terms were dropped from the charge balance equation. The [Cl⁻] term was added to account for the HCl added to Bioreactor 2.

All charge balances used K₀=1.70×10⁻³, K₁=4.98×10⁻⁷; K₂=5.77×10−11; and P=1.

Charge balances for the samples 1-8 used [Na⁺]=5.22×10⁻² and [Cl⁻]=0. Charge balances for the samples 9-16 used [Na⁺]=5.14×10⁻² and [Cl⁻]=1.49×10⁻². The parameter [NMA⁻] in equations for samples 9-16 was modified by a factor of 0.985 to account for its dilution by addition of hydrochloric acid.

Each of 16 equations has: [H⁺]=10^{-pH}, where pH is that measured for a data point; and yCO2% as specified by the bioreactor inlet gas flow rates for each data point.

The parameters m, K_{H}, and [NMA⁻] were then determined by minimizing the sum of each square times 10¹² for all charge balances. Parameters were found to be m=0.671; K_{H}=14.5 atm/(mol/L); [NMA⁻]=0.0327 mol/L. Each charge balance did not equate to exactly zero because the system of equation was overdetermined during this least-squares minimization. Hence the sum of squares for the parameter fitting was 5.84×10¹⁹.

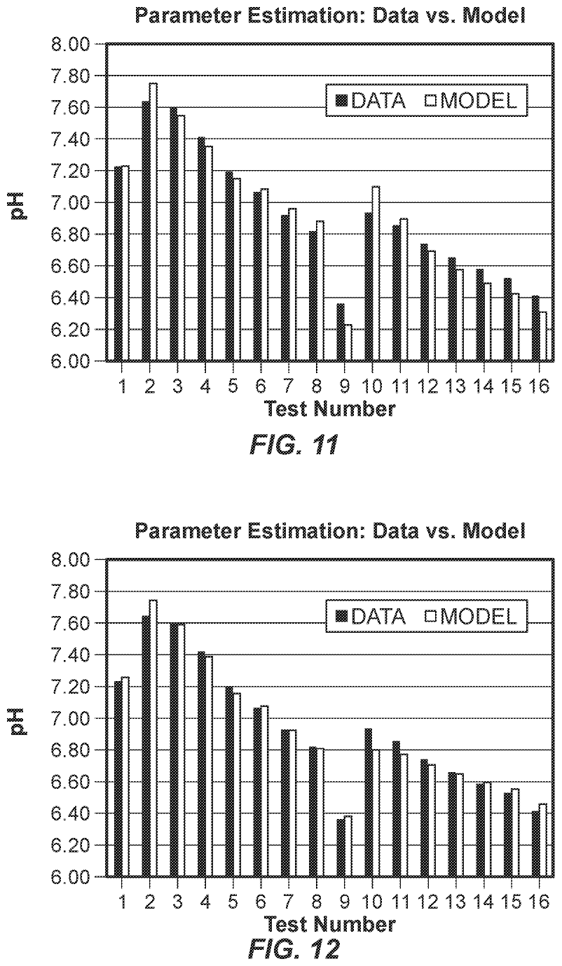

FIG. 11 shows the measured pH data and the model-fitted pH data (also shown in Table 4), calculated from the charge balances using the determined parameters.

The sum of squares for differences between data and model-fitted values was 0.108. When divided by 15 degrees of freedom, the estimate of model:data standard deviation was 0.085.

An alternate definition of [NMA⁻] was considered in a second analysis of the same data to consider the relationship between [NMA⁻] and pH. Here, [NMA⁻] was defined by:

[NMA⁻]=[C₀ₚ+C₁ₚ*(pH−7)]

where C₀ₚ and C₁ₚ are constants. The parameters determined by the least-squares method were m=1.254; K_{H}=85.75 atm/(mol/L); C₀ₚ=3.89×10⁻²; and C₁ₚ=1.37×10⁻². The sum of squares for the parameter fitting was 1.69×10¹⁹.

Table 5 and FIG. 12 show the model-fitted values for pH and the differences from these new values (considering [NMA⁻] as a function of pH. The sum of squares of for differences between data and model-fitted values was 0.0448. When divided by 15 degrees of freedom, the estimate of model:data standard deviation was 0.054.

TABLE 5

| | | | Measured | Modeled | |
|---|---|---|---|---|---|
| Sample | Bioreactor | yCO₂ % | pH | pH | Difference |
| 1 | 1 | 1 | 7.22 | 7.25 | 0.03 |
| 2 | 1 | 2 | 7.63 | 7.74 | 0.11 |
| 3 | 1 | 4 | 7.59 | 7.58 | 0.00 |
| 4 | 1 | 6 | 7.41 | 7.39 | −0.02 |
| 5 | 1 | 8 | 7.19 | 7.15 | −0.04 |
| 6 | 1 | 10 | 7.06 | 7.07 | 0.01 |
| 7 | 1 | 15 | 6.92 | 6.92 | 0.00 |
| 8 | 1 | 20 | 6.87 | 6.81 | −0.01 |
| 9 | 2 | 1 | 6.63 | 6.38 | 0.02 |
| 10 | 2 | 2 | 6.93 | 6.80 | −0.13 |
| 11 | 2 | 4 | 6.85 | 6.77 | −0.08 |
| 12 | 2 | 6 | 6.73 | 6.70 | −0.03 |
| 13 | 2 | 8 | 6.65 | 6.65 | 0.00 |
| 14 | 2 | 10 | 6.58 | 6.60 | 0.02 |
| 15 | 2 | 15 | 6.52 | 6.55 | 0.03 |
| 16 | 2 | 20 | 6.41 | 6.46 | 0.05 |

### Example 3

This example demonstrates how the charge balance model is used to determine a volume of sodium hydroxide to prepare a cell culture medium with 16 combinations of additional known species.

Specified with model parameters m=0.8412, K_{H}20.10 atm/(mol/L), and [NMA⁻]=2.89×10−mol/L, along with an initial volume of 5.3 mL NaOH used during preparation of the common basal Medium C, the charge balance model was used to estimate the volumes of sodium hydroxide required to be added to Medium C when 4 additional known components were added in various combinations, for a total of 16 distinct media preparations.

The 4 additional known components are glutamate, glutamine, asparagine, and ammonium chloride. The concentrations of all the anions and cations at equilibrium were determined using the pKa values. In the case of glutamic acid, which has three pKa values, the following dissociation/association equilibria were considered

$$[ {Glu}^{+} ] = \frac{[ Glu ] [ {H}^{+} ]}{{K}_{a 1}}$$

$$[ Gl {u}^{-} ] = \frac{{K}_{a 2} [ G l u ]}{[ {H}^{+} ]}$$

$$[ Gl {u}^{2 -} ] = \frac{{K}_{a 3} [ G l u ]}{[ {H}^{+} ]} = \frac{{K}_{a 3} {K}_{a 2} [ G l u ]}{{[ {H}^{+} ]}^{2}}$$

$$[ Glu ] = \frac{[ G l {u}_{0} ]}{1 + \frac{{K}_{a 2}}{[ {H}^{+} ]} + \frac{[ {H}^{+} ]}{{K}_{a 1}} + \frac{{K}_{a 2} {K}_{a 3}}{{[ {H}^{+} ]}^{2}}}$$

where [Glu₀] is the sum of the concentrations of glutamate and its ions added. The values of Kₐ₁, Kₐ₂, Kₐ₃ at 37° C. are 6.48×10⁻³ mol/L, 5.62×10⁻⁵ mol/L, and 2.14×10⁻¹⁰ mol/L, respectively.

In the case of glutamine, the following dissociation/association equilibria were considered

$$[ {Gln}^{+} ] = \frac{[ Gln ] [ {H}^{+} ]}{{K}_{a 1}}$$

$$[ Gl {n}^{-} ] = \frac{{K}_{a 2} [ Gln ]}{[ {H}^{+} ]}$$

$$[ Gln ] = \frac{[ G l {n}_{0} ]}{1 + \frac{{K}_{a 2}}{[ {H}^{+} ]} + \frac{[ {H}^{+} ]}{{K}_{a 1}}}$$

where [Gln₀] is the sum of the concentrations of glutamine and its ions added. The values of Kₐ₁ and Kₐ₂ at 37° C. are 6.76×10⁻³ mol/L and 6.76×10⁻¹⁹ mol/L, respectively. In the case of asparagine, the following dissociation/association equilibria were considered

$$[ A s {n}^{+} ] = \frac{[ A s n ] [ {H}^{+} ]}{{K}_{a 1}}$$

$$[ As {n}^{-} ] = \frac{{K}_{a 2} [ A s n ]}{[ {H}^{+} ]}$$

$$[ Asn ] = \frac{[ A s {n}_{0} ]}{1 + \frac{{K}_{a 2}}{[ {H}^{+} ]} + \frac{[ {H}^{+} ]}{{K}_{a 1}}}$$

where [Asn₀] is the sum of the concentrations of asparagine and its ions added. The values of Kₐ₁ and Kₐ₂ at 37° C. are 9.55×10⁻³ mol/L and 1.58×10⁻⁹ mol/L, respectively.

In the case of ammonia, ammonia comes from ammonium chloride salt

$$[ N {H}_{4} C l ] = [ {NH}_{3 , 0} ] + [ {Cl}^{-} ]$$

$$[ {NH}_{3 , 0} ] = [ {NH}_{3} ] + [ {NH}_{4}^{+} ]$$

$${K}_{b} = ( [ {NH}_{3 , 0} ] - [ {NH}_{4}^{+} ] ) * [ {H}^{+} ] / [ {NH}_{4}^{+} ]$$

$$[ {NH}_{4}^{+} ] = \frac{[ {NH}_{3 , 0} ]}{1 + \frac{{K}_{b}}{[ {H}^{+} ]}}$$

where [NH_{3,0}] is the sum of the concentrations of ammonia and its ions added. The value of K_{aA} at 37° C. is 5.75×10⁻¹⁰ mol/L.

The charge balance model is now written as:

[Na⁺]_{T}−[HCO₃⁻]−2*[CO₃²⁻]+[H⁺]−[OH⁻]−[NMA⁻]+[Gln⁺]−[Gln⁻]+[Asn⁺]−[Asn⁻]+[Glu⁺]−[Glu⁻]−2*[Glu²⁻]+[NH₄⁺]−[Cl⁻]=0

where [Na⁺]_{T} is the total amount of [Na⁺] coming from the recipe sodium bicarbonate, the recipe sodium hydroxide, and the additional amount of NaOH needed to achieve the desired pH. And [Cl⁻] is an added term to account for its presence in the ammonium reagent.

[Na⁺]_{T}=[Na⁺]_{recipe NaHCO}_{₃}+[Na⁺]_{recipe NaOH}+[Na⁺]_{Additional NaOH}

Using these equations, the amount of NaOH needed after specifying target process conditions of yCO₂%=5%, T=37° C., and pH=7.30 for each of the 16 different medium were calculated. Tables 6A and 6B (all concentrations in mol/L) show the concentration of components in the cell culture medium, including the amount of NaOH needed to be added.

TABLE 6A

| | [Na⁺] | [Na⁺] | | | | |
|---|---|---|---|---|---|---|
| | (recipe | (recipe | | | | |
| Medium | NaHCO₃) | NaOH) | [Gln₀] | [Asn₀] | [Glu₀] | [NH₄Cl] |
| Media C | 2.10E−02 | 2.60E−02 | 0.00E+00 | 0.00E+00 | 0.00E+00 | 0.00E+00 |
| 6G | 2.10E−02 | 2.60E−02 | 6.00E−03 | 0.00E+00 | 0.00E+00 | 0.00E+00 |
| 5A | 2.10E−02 | 2.60E−02 | 0.00E+00 | 5.00E−03 | 0.00E+00 | 0.00E+00 |
| 3Gu | 2.10E−02 | 2.60E−02 | 0.00E+00 | 0.00E+00 | 3.00E−03 | 0.00E+00 |
| 6G/5A | 2.10E−02 | 2.60E−02 | 6.00E−03 | 5.00E−03 | 0.00E+00 | 0.00E+00 |
| 6G/3Gu | 2.10E−02 | 2.60E−02 | 6.00E−03 | 0.00E+00 | 3.00E−03 | 0.00E+00 |
| 5A/3Gu | 2.10E−02 | 2.60E−02 | 0.00E+00 | 5.00E−03 | 3.00E−03 | 0.00E+00 |
| 6G/5A/3Gu | 2.10E−02 | 2.60E−02 | 6.00E−03 | 5.00E−03 | 3.00E−03 | 0.00E+00 |
| 6G/2Am | 2.10E−02 | 2.60E−02 | 6.00E−03 | 0.00E+00 | 0.00E+00 | 2.00E−03 |
| 5A/2Am | 2.10E−02 | 2.60E−02 | 0.00E+00 | 5.00E−03 | 0.00E+00 | 2.00E−03 |
| 3Gu/2Am | 2.10E−02 | 2.60E−02 | 0.00E+00 | 0.00E+00 | 3.00E−03 | 2.00E−03 |
| 6G/5A/2Am | 2.10E−02 | 2.60E−02 | 6.00E−03 | 5.00E−03 | 0.00E+00 | 2.00E−03 |
| 6G/3Gu/2Am | 2.10E−02 | 2.60E−02 | 6.00E−03 | 0.00E+00 | 3.00E−03 | 2.00E−03 |
| 5A/3Gu/2Am | 2.10E−02 | 2.60E−02 | 0.00E+00 | 5.00E−03 | 3.00E−03 | 2.00E−03 |
| 6G/5A/3Gu/2Am | 2.10E−02 | 2.60E−02 | 6.00E−03 | 5.00E−03 | 3.00E−03 | 2.00E−03 |
| 0G/0A/0Gu/2Am | 2.10E−02 | 2.60E−02 | 0.00E+00 | 0.00E+00 | 0.00E+00 | 2.00E−03 |

TABLE 6B

| | | | | | | [Na⁺] |
|---|---|---|---|---|---|---|
| | [Cl⁻] from | | | | | (Additional |
| Medium | Salt | [CO₂] | [NMA] | [H⁺] | [OH⁻] | NaOH) |
| Media C | 0.00E+00 | 1.93E−03 | 2.89E−02 | 5.06E−08 | 4.55E−07 | 0.00E+00 |
| 6G | 0.00E+00 | 1.93E−03 | 2.89E−02 | 5.07E−08 | 4.54E−07 | 1.00E−04 |
| 5A | 0.00E+00 | 1.93E−03 | 2.89E−02 | 5.08E−08 | 4.52E−07 | 2.00E−04 |
| 3Gu | 0.00E+00 | 1.93E−03 | 2.89E−02 | 5.05E−08 | 4.55E−07 | 3.10E−03 |
| 6G/5A | 0.00E+00 | 1.93E−03 | 2.89E−02 | 5.09E−08 | 4.52E−07 | 3.00E−04 |
| 6G/3Gu | 0.00E+00 | 1.93E−03 | 2.89E−02 | 5.06E−08 | 4.54E−07 | 3.20E−03 |
| 5A/3Gu | 0.00E+00 | 1.93E−03 | 2.89E−02 | 5.09E−08 | 4.52E−07 | 3.25E−03 |
| 6G/5A/3Gu | 0.00E+00 | 1.93E−03 | 2.89E−02 | 5.11E−08 | 4.50E−07 | 3.30E−03 |
| 6G/2Am | 2.00E−03 | 1.93E−03 | 2.89E−02 | 5.08E−08 | 4.53E−07 | 1.10E−04 |
| 5A/2Am | 2.00E−03 | 1.93E−03 | 2.89E−02 | 5.11E−08 | 4.50E−07 | 1.70E−04 |
| 3Gu/2Am | 2.00E−03 | 1.93E−03 | 2.89E−02 | 5.08E−08 | 4.53E−07 | 3.05E−03 |
| 6G/5A/2Am | 2.00E−03 | 1.93E−03 | 2.89E−02 | 5.13E−08 | 4.49E−07 | 2.20E−04 |
| 6G/3Gu/2Am | 2.00E−03 | 1.93E−03 | 2.89E−02 | 5.10E−08 | 4.51E−07 | 3.10E−03 |
| 5A/3Gu/2Am | 2.00E−03 | 1.93E−03 | 2.89E−02 | 5.13E−08 | 4.48E−07 | 3.15E−03 |
| 6G/5A/3Gu/2Am | 2.00E−03 | 1.93E−03 | 2.89E−02 | 5.15E−08 | 4.46E−07 | 3.20E−03 |
| 0G/0A/0Gu/2Am | 2.00E−03 | 1.93E−03 | 2.89E−02 | 5.07E−08 | 4.53E−07 | 0.00E+00 |

The concentration of Na⁺ from the recipe sodium bicarbonate and from the recipe NaOH are constant for all media and they are equal to 2.10E-02 M and 2.60E-02, respectively.

Each medium was a unique combination of all 16 possible combinations of the 4 components: glutamate, glutamine, asparagine, and ammonia. The starting concentrations for glutamine, asparagine, glutamate, and ammonia were 3.00E-03 M, 6.00E-03 M, 5.00E-03 M, and 2.00E-03 M, respectively. The concentration of Cl⁻ from ammonium chloride reagent used was 2.00E-03 M.

The concentration of CO₂ was calculated to be 1.93E-03 M and it was the same for all of the medium since the specified CO2 level was 5% at pressure of 1 atm.

The value of K_{H} used was 20.10 atm/(mol/L) and the value of m used was 0.8412. The Net Medium Acids (NMA) concentration was the same for all medium and equal to 2.89E-02 mol/L.

The expected concentration of H⁺ was calculated from the desired pH of 7.30 to be 5.01E-08 M, but the pH for each medium was not exactly at 7.30 because of rounding error. The concentration of OH⁻ ion was calculated from the concentration of H⁺ and was expected to be 4.59E-07 but again, it was close but not exactly equal due to rounding error. The K_{w} for water used in the calculation was 2.30E-14 M (since process condition temperature was set at 37 C).

The amount of NaOH needed for each medium was calculated in the last column of Table 6B using the charge balance equation.

With the amount of NaOH to be added to each cell medium to obtain the desired pH, as described above, the accuracy of the model was tested by preparing the culture media and measuring the pH. The 16 different media were placed into 250 ml shake flask an incubator at T=37° C. and CO2 setting of 5% after preparation. They were incubated to reach equilibrium overnight. Then, each flask removed one at a time from the incubator, sampled, and measured using the RapidLab BGA.

Due to CO₂ degassing that occurred during flask handing, sampling, and measurement, the dissolved CO₂ concentration was not that of equilibrium with 5% in the incubator. Thus, the pH of each flask was higher than the desired target. However, the RapidLab pCO₂ data was also collected for each sample, which provided a measured pCO₂ to evaluate the model (see Table 7). The measured pCO₂ values were used to estimate new yCO2% values for each flask. At steady state equilibrium, yCO2% =6.59×pCO₂ (mmHg)+4.65, an empirically determined equation for the RapidLab BGA. The target pH is not exactly 7.030 for each medium due to rounding of the volume of base to nearest microliter. The modeled pH and measured pH are shown in Table 7 and FIG. 13.

TABLE 7

| | | | | yCO2% | | |
|---|---|---|---|---|---|---|
| | Target | Target | Measured | from | Model pH at | Measured |
| Medium | yCO₂% | pH | pCO₂ | correlation | yCO2% corr | pH |
| Custom Medium C | 5.0 | 7.296 | 34.4 | 4.51 | 7.333 | 7.326 |
| 6G | 5.0 | 7.295 | 34.1 | 4.47 | 7.336 | 7.331 |
| 5A | 5.0 | 7.294 | 34.2 | 4.48 | 7.333 | 7.329 |
| 3Gu | 5.0 | 7.296 | 33.5 | 4.38 | 7.345 | 7.336 |
| 6G/5A | 5.0 | 7.293 | 32.5 | 4.23 | 7.353 | 7.345 |
| 6G/3Gu | 5.0 | 7.296 | 32.3 | 4.20 | 7.359 | 7.349 |
| 5A/3Gu | 5.0 | 7.293 | 31.8 | 4.12 | 7.362 | 7.359 |
| 6G/5A/3Gu | 5.0 | 7.291 | 31.1 | 4.01 | 7.369 | 7.365 |
| 6G/2Am | 5.0 | 7.294 | 30.6 | 3.94 | 7.380 | 7.367 |
| 5A/2Am | 5.0 | 7.292 | 30.6 | 3.94 | 7.377 | 7.379 |
| 3Gu/2Am | 5.0 | 7.294 | 29.9 | 3.83 | 7.391 | 7.390 |
| 6G/5A/2Am | 5.0 | 7.290 | 30.0 | 3.85 | 7.383 | 7.386 |
| 6G/3Gu/2Am | 5.0 | 7.292 | 30.0 | 3.85 | 7.387 | 7.382 |
| 5A/3Gu/2Am | 5.0 | 7.290 | 28.8 | 3.66 | 7.401 | 7.399 |
| 6G/5A/3Gu/2Am | 5.0 | 7.288 | 28.4 | 3.60 | 7.404 | 7.405 |
| 0G/0A/0Gu/2Am | 5.0 | 7.295 | 28.1 | 3.56 | 7.418 | 7.415 |

The estimated sigma value was 0.006. This is within the precision of a single BGA pH reading, which is 0.01-0.02. Thus, even though there was a measurement difficulty with the CO₂ degassing, the pH of the solution was that expected from the model once the actual yCO2% (as estimated from the measured pCO₂) was input into the model calculation.

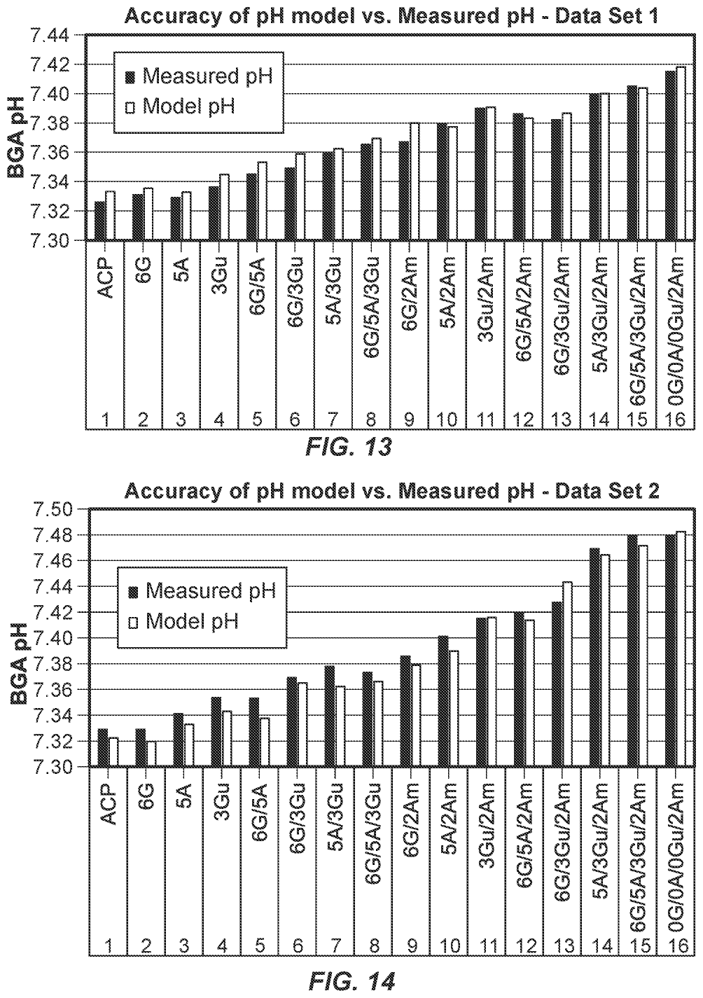

A second set of 16 solutions was prepared on a different day, and the accuracy assessment repeated. See Table 8 and FIG. 14 The pH were higher than target again because of degassing of CO₂, but the measured pH and model-calculated pH at the measured pCO₂ were very similar, in this case with a sigma=0.009.

TABLE 8

| | | | | yCO2% | | |
|---|---|---|---|---|---|---|
| | Target | Target | Measured | from | Model pH at | Measured |
| Medium | yCO2% | pH | pCO2 | correlation | yCO2% corr | pH |
| ACP | 5.0 | 7.296 | 35.3 | 4.65 | 7.322 | 7.329 |
| 6G | 5.0 | 7.295 | 35.4 | 4.67 | 7.320 | 7.329 |
| 5A | 5.0 | 7.294 | 34.2 | 4.48 | 7.333 | 7.341 |
| 3Gu | 5.0 | 7.296 | 33.6 | 4.39 | 7.343 | 7.354 |
| 6G/5A | 5.0 | 7.293 | 33.7 | 4.41 | 7.338 | 7.353 |
| 6G/3Gu | 5.0 | 7.296 | 31.8 | 4.12 | 7.365 | 7.369 |
| 5A/3Gu | 5.0 | 7.293 | 31.8 | 4.12 | 7.362 | 7.378 |
| 6G/5A/3Gu | 5.0 | 7.291 | 31.3 | 4.04 | 7.367 | 7.373 |
| 6G/2Am | 5.0 | 7.294 | 30.7 | 3.95 | 7.379 | 7.386 |
| 5A/2Am | 5.0 | 7.292 | 29.7 | 3.80 | 7.390 | 7.401 |
| 3Gu/2Am | 5.0 | 7.294 | 28.2 | 3.57 | 7.416 | 7.415 |
| 6G/5A/2Am | 5.0 | 7.290 | 27.9 | 3.53 | 7.414 | 7.419 |
| 6G/3Gu/2Am | 5.0 | 7.292 | 26.3 | 3.28 | 7.443 | 7.428 |
| 5A/3Gu/2Am | 5.0 | 7.290 | 24.8 | 3.06 | 7.465 | 7.469 |
| 6G/5A/3Gu/2Am | 5.0 | 7.288 | 24.2 | 2.97 | 7.472 | 7.479 |
| 0G/0A/0Gu/2Am | 5.0 | 7.29′5 | 24.3 | 2.98 | 7.482 | 7.479 |

### Example 4

This example demonstrates the accuracy of the charge balance model in achieving target pH for Medium B by comparing the pH of the prepared solutions against the model values for the RapidLab (pH=7.04) and the NOVA Flex II (pH=7.15).

Medium B was prepared according to the manufacturer procedure and then pumped into the bioreactor in a sterile manner. The bioreactor was then set at 37° C. and the gas flow was set to achieve a 10% CO₂ level. Steady state was allowed to reach after the online pH was stable for at least 15 minutes. The sample was then taken to be measured on both the RapidLab and the NOVA Flex II devices. The experiment was repeated 16 times using the same or different bioreactor, on the same or different days, and using the same or different lots of Medium B.

The concentration of NMA was calculated to be 3.40E-02 mol/L for Medium B. The m and the K_{H} values for the pH model based on the RapidLab device were 0.8412 and 20.85 atm/(mol/L), respectively. The m and the K_{H} values for the pH model based on the NOVA Flex II device were 0.9128 and 31.78 atm/(mol/L), respectively.

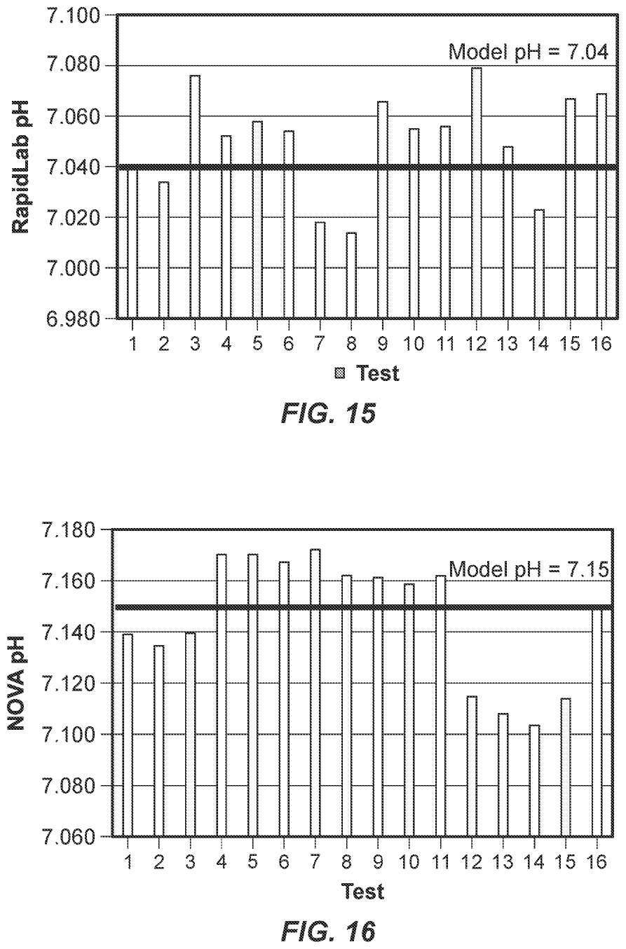

The measured pH using the RapidLab device of all 16 tests are plotted in FIG. 15. The measured data was noisy and deviated from the expected model pH for this device (pH=7.04) could be due to the noise from the single reading of each measurement. Also, each mass flow controller (MFC) that controls the gas flow rate of each bioreactor has noise associated with it that have not provide CO₂ mole percentage of exactly at 10%. Lastly, each batch of medium was prepared slightly different due to measurement noise also may have contributed to the observed varied pH. However, the average pH measured by the RapidLab (n=16) was found to be 7.051, with the standard deviation of 0.020. The 95% confidence limit was calculated to be 0.011. The difference of the mean pH from the pH target of 7.04 was not statistically significant. Thus, the pH model for Medium B and the RapidLab device accurately predicted the pH of the prepared Medium B as measured using the RapidLab device.

The measured pH using the Nova flex II device of all 16 tests are plotted in FIG. 16. That the measured data was again noisy and deviated from the expected model pH for this device (pH=7.15) could be due to similar reasons described above. The average pH measured by the Nova flex II (n=16) was found to be 7.145, with the standard deviation of 0.040. The 95% confidence limit was calculated to be 0.013. The difference of the mean pH from the pH target of 7.15 was not statistically significant. Thus, the pH model for Medium B and the NOVA Flex II device accurately predicted the pH of the prepared Medium B as measured using the NOVA Flex II device.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method of adjusting the pH of a cell culture medium, comprising:
obtaining, for the cell culture medium, a functional relationship between a concentration of dissolved carbon dioxide in the cell culture medium and a mole fraction of gaseous carbon dioxide applied to the cell culture medium, and a concentration of net medium acids in the cell culture medium;
adding carbonate salt or bicarbonate salt to the cell culture medium to obtain a desired carbonate salt or bicarbonate salt concentration in the cell culture medium; and
determining, using a charge balance model, an amount of strong acid or strong base to be added to the cell culture medium to adjust the pH of the cell culture medium to a desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH.

2. The method of claim 1, further comprising adding the determined amount of strong acid or strong base to the cell culture medium, thereby making a pH-adjusted cell culture medium.

3. The method of claim 1, wherein the carbonate salt or the bicarbonate salt is sodium carbonate or sodium bicarbonate.

4. The method of claim 1, further comprising supplementing the cell culture medium with one or more ionic compounds, wherein the charge balance model is further based on the concentration of the one or more ionic compounds.

5. The method of claim 4, wherein the one or more ionic compounds comprises one or more amino acids or ammonium chloride.

6. The method of claim 5, wherein the one or more amino acids comprises glutamine, asparagine, or glutamic acid.

7. The method of claim 1, wherein the strong base is sodium hydroxide.

8. The method of claim 1, wherein the strong acid is hydrochloric acid.

9. The method of claim 1, wherein the charge balance model is defined by:
[Na⁺]−[HCO₃⁻]−2*[CO₃²⁻]+[H⁺]−[OH⁻]−[NMA⁻]−[A⁻]+[B⁺]=0
wherein:
[Na⁺] is a concentration of sodium ions added as sodium hydroxide, sodium bicarbonate, or sodium carbonate to the cell culture medium;
[H⁺] is a concentration of protons in the cell culture medium needed to obtain the desired pH;
[OH⁻] is a concentration of hydroxide anions in the cell culture medium;
[NMA⁻] is the concentration of net medium acid ions in the cell culture medium;
[A⁻] is a concentration of negatively charged ions added to the cell culture medium, multiplied by the absolute value of their charge, excluding any OH⁻ or negatively charged ions included in [NMA−]; and
[B⁺] is a concentration of positively charged ions added to the cell culture medium, multiplied by the absolute value of their charge, excluding any H⁺, sodium ions included in [Na⁺], or positively charged ions included in [NMA−].

10. The method of claim 9, wherein:
$$[ {HCO}_{3}^{-} ] = \frac{{K}_{0} * {K}_{1} * P * {( {y CO}_{2} )}^{m}}{100 * {K}_{H} * [ {H}^{+} ]}$$
$$[ {CO}_{3}^{2 -} ] = \frac{{K}_{0} * {K}_{1} * {K}_{2} * P * {( {y CO}_{2} )}^{m}}{100 * {K}_{H} * {[ {H}^{+} ]}^{2}}$$
wherein:
K₀, K₁, and K₂ are dissociation constants for bicarbonate and carbonate anions;
P is a gas pressure applied to the cell culture medium;
yCO₂ is a molar percentage of CO₂ gas phase applied to the cell culture medium; and
m and K_{H} are each empirically determined parameters for the cell culture medium.

11. (canceled)

12. (canceled)

13. The method of claim 12, wherein the concentration of net medium acids in the cell culture medium is modeled as:
[NMA⁻]=[C₀ₚ+C₁ₚ*(pH−7)]
wherein:
[NMA⁻] is the concentration of net medium acid ions in the cell culture medium; and
C₀ₚ and C₁ₚ are each empirically determined constants for the cell culture medium.

14. The method of claim 1, wherein the concentration of net medium acids in the cell culture medium is modeled in the charge balance model as a function of temperature.

15. The method of claim 1, wherein the concentration of net medium acids in the cell culture medium is modeled in the charge balance model as a function of pH and temperature.

16. (canceled)

17. (canceled)

18. (canceled)

19. (canceled)

20. (canceled)

21. (canceled)

22. A method of culturing cells, comprising:
adjusting the pH of a cell culture medium according to the method of claim 1; and
culturing cells in the pH-adjusted cell culture medium.

23. The method of claim 22, wherein the cells are mammalian cells.

24. The method of claim 22, wherein cells are Chinese hamster ovary (CHO) cells.

25. (canceled)

26. (canceled)

27. (canceled)

28. (canceled)

29. (canceled)

30. A system, comprising:
one or more processors; and
a memory communicatively coupled to the one or more processors and configured to store instructions that, when executed by the one or more processors, cause the system to:
receive, at the one or more processors, for a cell culture medium, one or more parameters indicating a functional relationship between a concentration of dissolved carbon dioxide in the cell culture medium and a mole fraction of gaseous carbon dioxide applied to the cell culture medium, and a net medium acids parameter indicating a concentration of net medium acids in the cell culture medium;
receive, at the one or more processors, a carbonate salt or bicarbonate salt parameter indicating a desired carbonate salt or bicarbonate salt concentration in the cell culture medium;
receive, at the one or more processors, a pH parameter indicating a desired pH of the cell culture medium; and
determine, using a charge balance model, an amount of strong acid or strong base to be added to the cell culture medium to adjust the pH of the cell culture medium to the desired pH, wherein the charge balance model is based on at least the functional relationship between the concentration of dissolved carbon dioxide in the cell culture medium and the mole fraction of gaseous carbon dioxide applied to the cell culture medium, the concentration of net medium acids in the cell culture medium, the desired carbonate salt or bicarbonate salt concentration in the cell culture medium, and the desired pH.

31. A method for producing a polypeptide in a host cell expressing the polypeptide, comprising culturing the host cell in a cell culture medium by preparing a cell culture medium with sodium bicarbonate to tightly control the pH of the medium, comprising:
determining the excipients and relative amounts to be added to a cell culture medium to define a recipe,
preparing a solution using the recipe and determining the pH of the solution to define a first data set;
placing the solution in a CO₂ gassed and agitated bioreactor and allowing it to equilibrate to determine the resulting pH and pCO₂ values to define a second data set;
placing the solution in an incubator at a defined temperature and molar percent CO₂ and determining the pH and pCO₂ measurements to define a third data set;
using the first, second, and third data sets and a pH model according to:
$$pH - {pK}_{a} - log [ B ] = log ( \frac{1}{s} ) - m * log ( {y}_{C {O}_{2}} )$$
to solve for the parameter values of m, s, and net medium acids simultaneously by minimizing:
Σ(k*[C^{k}])=0;
defining a target pH for the cell culture medium and adding an appropriate concentration of base to the cell culture medium as determined from the pH model to achieve the pH equivalence wherein the cell culture medium pH is tightly controlled; and
producing the polypeptide.

32. (canceled)

33. The method of claim 31, wherein the excipients are selected from the group consisting of glutamine, glutamate, asparagine, ammonium chloride, sodium chloride, and sodium hydroxide.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)
